(19) United States
(12) Patent Application Publication
(10) Pub. No.: US 2005/0232924 A1
(43) Pub. Date: Oct. 20, 2005

(54) Antibodies and/or conjugates thereof which bind to the amino terminal fragment of urokinase, compositions and uses thereof

(75) Inventors: Andrew P. Mazar, San Diego, CA (US); Robert J. Ternansky, San Diego, CA (US); Graham Parry, San Diego, CA (US); Patricia L. Gladstone, San Diego, CA (US); Susan Gawlak, Hamden, CT (US)

Correspondence Address: Sunil K. Singh;Dorsey & Whitney LLP, Intellectual Property Department, Four Embarcadero Center, Suite 3400, San Francisco, CA, 94111-4187, US

(73) Assignee: Attenuon LLC

(21) Appl. No.: 10/993,007

(22) Filed: Nov. 18, 2004

Related U.S. Application Data
(60) Provisional application No. 60/523,255, filed on Nov. 18, 2003.

(51) Int. Cl. C07K 16/40; A61K 39/395

(52) U.S. Cl. 424/146.1; 530/388.26

(57) ABSTRACT

Antibodies and/or conjugates thereof which bind to the amino terminal fragment of urokinase, compositions and uses thereof are provided. The antibodies and antibody conjugates, which may include a therapeutic agent or a diagnostic agent, may be used to treat, prevent or detect diseases such as for example cancer.

### 1. FIELD

Antibodies and/or conjugates thereof which bind to the amino terminal fragment of urokinase, compositions and uses thereof are provided. More specifically, antibodies and/or conjugates thereof which may bind the Kringle region, the growth factor domain region or the C-terminal region of the amino terminal fragment of urokinase, compositions and uses thereof are provided. The antibodies and antibody conjugates, which may include a therapeutic agent or a diagnostic agent may be used to treat, prevent or detect diseases such as, for example, cancer.

### 2. BACKGROUND

The urokinase plasminogen activator system, comprised of the serine protease urokinase (uPA), the urokinase cell surface receptor (uPAR) and plasminogen activator inhibitor-1 (PAI-1), is one of the factors responsible for neo-vascularization, invasion and metastasis of many solid tumors (Danø et al., Adv. Cancer Res., 1985, 44:139-266). uPAR plays an essential role in the regulated degradation and remodeling of the extracellular matrix by tumor cells and angiogenic endothelial cells (FIG. 1). uPA-uPAR dependent cascades also result in the activation of promatrix metalloproteinase-9 and the activation and release of growth factors and angiogenic factors including HGF, VEGF and TGFβ.

Cells produce uPA in an inactive form as a 411 amino acid protein, pro-urokinase (pro-uPA) or single-chain uPA (scuPA), which then binds to uPAR. This binding event is a prerequisite for the efficient activation of scuPA to two-chain uPA (tcuPA) in a cellular milieu (Ellis et al., J. Biol. Chem. 1989, 264:2185-88). Pro-uPA is activated by a single proteolytic cleavage between amino acid 158 (Lys) and 159 (Ile) to activate the proenzyme. Cleavage results in the formation of the two-chain active uPA (tcuPA) which results in a conformational change and in the gain of plasminogen activator activity both with natural and synthetic substrates. uPA is a three-domain protein comprising an N-terminal growth factor domain, a Kringle domain, and (3) a C-terminal serine protease domain. uPAR, the receptor for pro-uPA, is also a multi-domain protein anchored by a glycosyl-phosphatidylinositol anchor to the outer leaf of the cell membrane (Behrendt et al., Biol. Chem. Hoppe-Seyler 1995, 376:269-279).

uPAR is not usually expressed at detectable levels on quiescent cells and must therefore be upregulated before activities of the uPA system are initiated. uPAR expression is stimulated in vitro by agents such as phorbol esters (Lund et al., J. Biol. Chem. 1991, 266:5177-5181), the transformation of epithelial cells and various growth factors and cytokines such as VEGF, bFGF, HGF, IL-1, TNFα, (in endothelial cells) and GM-CSF (in macrophages) (Mignatti et al., J. Cell Biol. 1991, 113:1193-1201; Mandriota et al., J. Biol. Chem. 270:9709-9716; Yoshida et al., Inflammation 1996, 20:319-326). The uPAR expression has the functional consequence of increasing cell motility, invasion, and adhesion (Mandriota et al., supra). More importantly, uPAR appears to be up-regulated in vivo in most human carcinomas examined to date, specifically, in the tumor cells themselves, in tumor-associated endothelial cells undergoing angiogenesis and in macrophages (Pyke et al., Cancer Res. 1993, 53:1911-15) which may participate in the induction of tumor angiogenesis (Lewis et al., J. Leukoc. Biol. 1995, 57:747-751). uPAR expression in cancer patients is present in advanced disease and has been correlated with a poor prognosis in numerous human carcinomas (Hofmann et al., Cancer 1996, 78:487-92; Heiss et al., Nature Med. 1995, 1:1035-39). Moreover, uPAR is not expressed uniformly throughout a tumor but tends to be associated with the invasive margin and is considered to represent a phenotypic marker of metastasis in human gastric cancer. Accordingly, uPAR is essential in the regulated degradation and remodeling of the extracellular matrix by tumor cells and angiogenic endothelial cell (FIG. 1). The important role of uPA-uPAR in tumor growth and its abundant expression within tumor, but not normal tissue, makes this system an attractive diagnostic and therapeutic target.

Therapeutic use of uPA, pro-uPA, tissue plasminogen activator (tPA) or streptokinase (for thromboembolism) has been investigated for the treatment of pathological states, such as cancer. However, these therapeutic agents require very high doses due, in part, to rapid clearance. Possible reasons for the short half-life of these proteins include binding to specific circulating inhibitors, binding to receptors, internalization and degradation of inhibitor-bound and/or receptor-bound PA.

Accordingly, what is needed are novel therapeutic and diagnostic agents that can treat, and/or prevent diseases mediated by the uPA-uPAR system.

### 3. SUMMARY

These and other needs are satisfied by providing antibodies and/or conjugates thereof which bind to the amino terminal fragment of urokinase, compositions and uses thereof. The antibodies and antibody conjugates, which may include a therapeutic agent or a diagnostic agent can be used to treat, prevent or detect diseases such as, for example, cancer.

In one aspect, an antibody which binds to the amino terminal fragment of urokinase is provided. In some embodiments, the antibody binds to the growth factor domain of urokinase. In other embodiments, the antibody binds to the Kringle domain of urokinase.

The antibody, which is preferably a monoclonal antibody, may be internalized into a cell after binding urokinase. In some embodiments, the antibody is fused to a protein toxin. In other embodiments, the antibody is conjugated to a therapeutic agent. Preferably, the therapeutic agent is a cytotoxic cancer agent such as a taxane, a camptothecin or an epithilone. In some embodiments, the therapeutic agent is doxorubicin. In other embodiments, the therapeutic agent is a radionuclide. In still other embodiments, the antibody is conjugated to a diagnostic agent which may be a radionuclide, a agent imageable by positron emission tomography, an agent imageable by magnetic resonance a fluorescent agent, a fluorogen, a chromophore, a chromogen, a phosphorescent agent, a chemiluminescent agent or a bioluminescent agent.

In another aspect, pharmaceutical compositions are provided which generally comprise one or more antibodies or conjugates thereof and a pharmaceutically acceptable vehicle such as a diluent, carrier, excipient or adjuvant. The choice of diluent, carrier, excipient and adjuvant will depend upon, among other factors, the desired mode of administration.

In still another aspect, diagnostic compositions are provided which generally comprise one or more antibodies or conjugates thereof and a pharmaceutically acceptable vehicle such as a diluent, carrier, excipient or adjuvant. The choice of diluent, carrier, excipient and adjuvant will depend upon, among other factors, the desired mode of administration.

In still another aspect, methods for inhibiting cell migration, cell invasion, cell proliferation or angiogenesis are provided. The methods generally comprise contacting cells with an effective amount of an antibody and/or conjugate thereof and/or pharmaceutical compositions thereof.

In still another aspect, methods for treating or preventing cell migration, cell invasion, cell proliferation or angiogenesis are provided. The methods generally involve administering to a patient in need of such treatment or prevention a therapeutically effective amount of an antibody and/or conjugate thereof and/or pharmaceutical compositions thereof.

In still another aspect, methods for inducing apoptosis are provided. The methods generally comprise contacting cells with an effective amount of an antibody and/or conjugate thereof and/or pharmaceutical compositions thereof.

In still another aspect, methods for inducing apoptosis are provided. The methods generally involve administering to a patient in need of such treatment or prevention a therapeutically effective amount of an antibody and/or conjugate thereof and/or pharmaceutical compositions thereof.

In still another aspect, methods for treating or preventing a disease caused by cell migration, cell invasion, cell proliferation or angiogenesis are provided. The methods generally involve administering to a patient in need of such treatment or prevention a therapeutically effective amount of an antibody and/or conjugate thereof and/or pharmaceutical compositions thereof.

In still another aspect, methods for detecting cell migration, cell invasion, cell proliferation or angiogenesis are provided. The methods generally comprise contacting cells with an effective amount of an antibody and/or conjugate thereof and/or diagnostic compositions thereof.

In still another aspect, methods for detecting cell migration, cell invasion, cell proliferation or angiogenesis are provided. The methods generally involve administering to a patient in need of such treatment or prevention a diagnostically effective amount of an antibody and/or conjugate thereof and/or diagnostic compositions thereof.

In still another aspect, methods for detecting a disease caused by cell migration, cell invasion, cell proliferation or angiogenesis are provided. The methods generally involve administering to a patient in need of such treatment or prevention a diagnostically effective amount of an antibody and/or conjugate thereof and/or diagnostic compositions thereof.

In still another aspect, methods for detecting whether a antibody and/or conjugate thereof which binds to the amino terminal fragment of urokinase is internalized into a cell are provided. In one embodiment, the cell is contacted with the antibody and/or conjugate thereof and then washed, fixed and permeabilized. Then a diagnostically labeled secondary antibody is added and the diagnostic label is detected. In another embodiment, the antibody and/or conjugate thereof is diagnostically labeled. The cell is contacted with the diagnostically labeled antibody and/or conjugate thereof and the diagnostic label is detected.

### 4. BRIEF DESCRIPTION OF THE DRAWINGS

FIG. 1 illustrates the role of uPAR in regulating degradation and remodeling of the extracellular matrix by tumor cells and angiogenic endothelial cells;

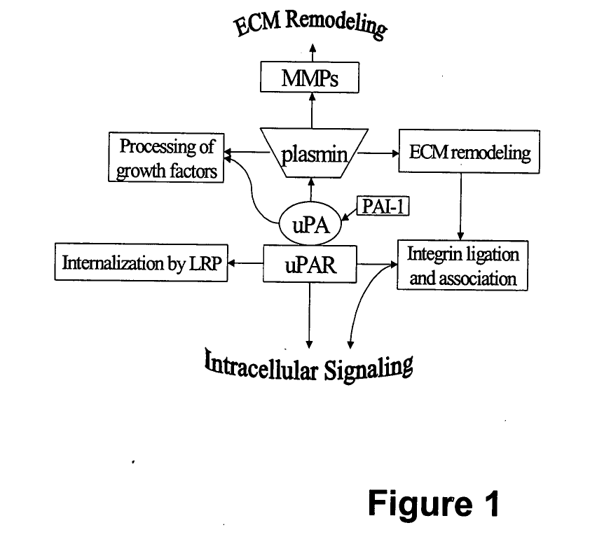

FIG. 2 illustrates the primary sequence of mature urokinase;

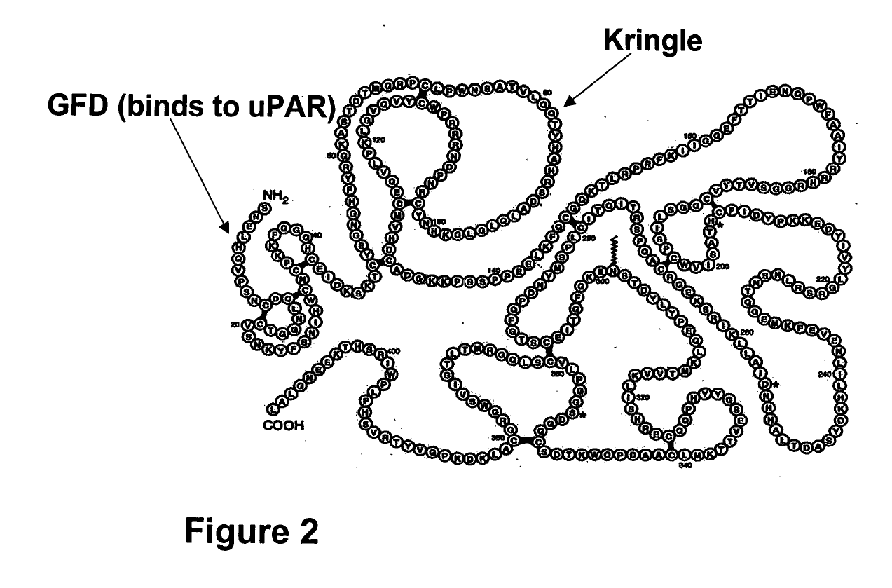

FIG. 3 illustrates epitope mapping of monoclonal antibodies ATN-291 and ATN-292;

FIG. 4 illustrates binding of ATN-291 and ATN-292 to immobilized urokinase;

FIG. 5 illustrates inhibition of binding of ¹²⁵I labeled amino terminal fragment of urokinase to HeLa cells;

FIG. 6 illustrates inhibition of tumor growth by ATN-291 and ATN-292;

FIG. 7 illustrates binding of [¹²⁵I]-ATN-291 to receptor bound urokinase;

FIG. 8 illustrates internalization of [¹²⁵I]-ATN-291 by MDA-MB-231 cells;

FIG. 9 illustrates internalization of ATN-291 by MDA-MB-231 cells;

FIG. 10 illustrates internalization of ATN-291 -CY5 conjugates by MDA-MB-231 cells;

FIG. 11 illustrates conjugation of doxorubicin to ATN-291;

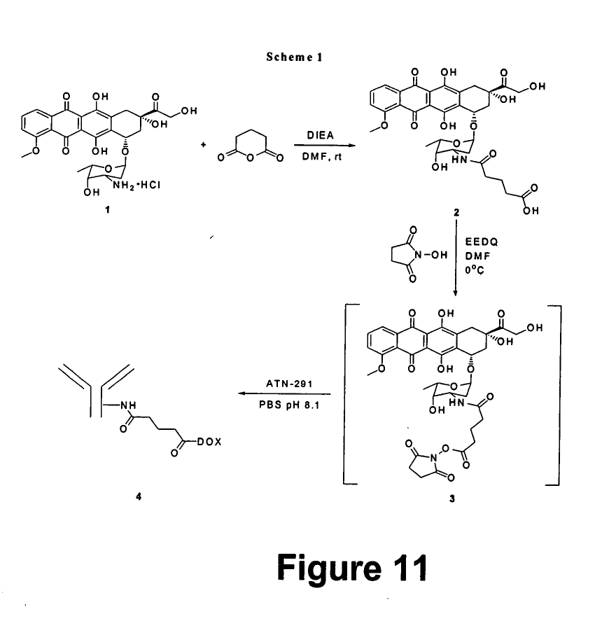

FIG. 12 illustrates direct binding of ATN-291-Dox conjugate to immobilized urokinase;

FIG. 13 illustrates ATN-291-Dox conjugate inhibits cell proliferation of MDA-MB-231 cells; and

FIG. 14 illustrates internalization of ATN-291 -Dox conjugates by MDA-MB-231 cells.

### 5. DETAILED DESCRIPTION

### 5.1 Definitions

“Alkyl” by itself or as part of another substituent refers to a saturated or unsaturated, branched, straight-chain or cyclic monovalent hydrocarbon radical derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl (allyl), cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like.

The term “alkyl” is specifically intended to include groups having any degree or level of saturation, i.e., groups having exclusively single carbon-carbon bonds, groups having one or more double carbon-carbon bonds, groups having one or more triple carbon-carbon bonds and groups having mixtures of single, double and triple carbon-carbon bonds. Where a specific level of saturation is intended, the expressions “alkanyl,” “alkenyl,” and “alkynyl” are used. Preferably, an alkyl group comprises from 1 to 20 carbon atoms, more preferably, from 1 to 10 carbon atoms, most preferably, from 1 to 6 carbon atoms.

“Alkanyl” by itself or as part of another substituent refers to a saturated branched, straight-chain or cyclic alkyl radical derived by the removal of one hydrogen atom from a single carbon atom of a parent alkane. Typical alkanyl groups include, but are not limited to, methanyl; ethanyl; propanyls such as propan-1-yl, propan-2-yl (isopropyl), cyclopropan-1-yl, etc.; butanyls such as butan-1-yl, butan-2-yl (sec-butyl), 2-methyl-propan-1-yl (isobutyl), 2-methyl-propan-2-yl (t-butyl), cyclobutan-1-yl, etc.; and the like.

“Alkenyl” by itself or as part of another substituent refers to an unsaturated branched, straight-chain or cyclic alkyl radical having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like.

“Alkynyl” by itself or as part of another substituent refers to an unsaturated branched, straight-chain or cyclic alkyl radical having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like.

“Aryl” by itself or as part of another substituent refers to a monovalent aromatic hydrocarbon radical derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. Preferably, an aryl group comprises from 6 to 20 carbon atoms, more preferably, from 6 to 12 carbon atoms.

“Arylalkyl” by itself or as part of another substituent refers to an acyclic alkyl radical in which one of the hydrogen atoms bonded to a carbon atom, typically a terminal or sp3 carbon atom, is replaced with an aryl group. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl and/or arylalkynyl is used. Preferably, an arylalkyl group is (C₇-C₃₀) arylalkyl, e.g., the alkanyl, alkenyl or alkynyl moiety of the arylalkyl group is (C₁-C₁₀) and the aryl moiety is (C₆-C₂₀), more preferably, an arylalkyl group is (C₇-C₂₀) arylalkyl, e.g., the alkanyl, alkenyl or alkynyl moiety of the arylalkyl group is (C₁-C₈) and the aryl moiety is (C₆-C₁₂).

“Heteroalkyl, Heteroalkanyl, Heteroalkenyl and Heteroalkynyl” by themselves or as part of another substituent refer to alkyl, alkanyl, alkenyl and alkynyl groups, respectively, in which one or more of the carbon atoms (and any associated hydrogen atoms) are independently replaced with the same or different heteroatomic groups. Typical heteroatomic groups which can be included in these groups include, but are not limited to, —O—, —S—, —O—O—, —S—S—, —O—S—, —NR³⁷R³⁸—, ═N—N═, —N═N—, —N═N—NR³⁹R⁴⁰, —PR⁴¹—, —P(O)₂—, —POR⁴²—, —O—P(O)₂—, —SO—, —SO₂—, —SnR⁴³R⁴⁴— and the like, where R³⁷, R³⁸, R³⁹, R⁴⁰, R⁴¹, R⁴², R⁴³ and R⁴⁴ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl or substituted heteroarylalkyl.

“Heteroaryl” by itself or as part of another substituent, refers to a monovalent heteroaromatic radical derived by the removal of one hydrogen atom from a single atom of a parent heteroaromatic ring system. Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. Preferably, the heteroaryl group is from 5-20 membered heteroaryl, more preferably from 5-10 membered heteroaryl. Preferred heteroaryl groups are those derived from thiophene, pyrrole, benzothiophene, benzofuran, indole, pyridine, quinoline, imidazole, oxazole and pyrazine.

“Heteroarylalkyl” by itself or as part of another substituent refers to an acyclic alkyl radical in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylalkenyl and/or heterorylalkynyl is used. In preferred embodiments, the heteroarylalkyl group is a 6-30 membered heteroarylalkyl, e.g., the alkanyl, alkenyl or alkynyl moiety of the heteroarylalkyl is 1-10 membered and the heteroaryl moiety is a 5-20-membered heteroaryl, more preferably, 6-20 membered heteroarylalkyl, e.g., the alkanyl, alkenyl or alkynyl moiety of the heteroarylalkyl is 1-8 membered and the heteroaryl moiety is a 5-12-membered heteroaryl.

“Parent Aromatic Ring System” refers to an unsaturated cyclic or polycyclic ring system having a conjugated π electron system. Specifically included within the definition of “parent aromatic ring system” are fused ring systems in which one or more of the rings are aromatic and one or more of the rings are saturated or unsaturated, such as, for example, fluorene, indane, indene, phenalene, etc. Typical parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like.

“Parent Heteroaromatic Ring System” refers to a parent aromatic ring system in which one or more carbon atoms (and any associated hydrogen atoms) are independently replaced with the same or different heteroatom. Typical heteroatoms to replace the carbon atoms include, but are not limited to, N, P, O, S, Si, etc. Specifically included within the definition of “parent heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, xanthene, etc. Typical parent heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like.

“Diagnostically effective amount” refers to the amount of an antibody that, when administered to a patient for detection of a disease, is sufficient to detect the disease. The “diagnostically effective amount” will vary depending on the compound, the disease and its severity and the age, weight, etc., of the patient to be treated.

“Effective amount” refers to the amount of an antibody that, when administered for example, to detect, induce or inhibit a particular property or condition is sufficient to detect the to detect, induce or inhibit the property or condition. The “effective amount” will vary depending on the antibody and the particular property or condition.

“Patient” includes humans. The terms “human” and “patient” are used interchangeably herein.

“Pharmaceutically acceptable salt” refers to a salt of an antibody, which is pharmaceutically acceptable and possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like.

“Pharmaceutically acceptable vehicle” refers to a diluent, adjuvant, excipient or carrier with which an antibody and/or conjugate thereof is administered.

“Preventing” or “prevention” refers to a reduction in risk of acquiring a disease or disorder (i.e., causing at least one of the clinical symptoms of the disease not to develop in a patient that may be exposed to or predisposed to the disease but does not yet experience or display symptoms of the disease).

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the patient. In yet another embodiment, “treating” or “treatment” refers to inhibiting the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

“Therapeutically effective amount” means the amount of an antibody and/or conjugate thereof that, when administered to a patient for treating a disease, is sufficient to effect such treatment for the disease. The “therapeutically effective amount” will vary depending on the compound, the disease and its severity and the age, weight, etc., of the patient to be treated.

Reference will now be made in detail to embodiments of the invention. While the invention will be described in conjunction with these embodiments, it will be understood that it is not intended to limit the invention to those embodiments. To the contrary, it is intended to cover alternatives, modifications, and equivalents as may be included within the spirit and scope of the invention as defined by the appended claims.

### 5.2 Antibodies

Antibodies and/or conjugates thereof which bind to the amino terminal fragment of urokinase. The antibodies and/or conjugates thereof may bind the Kringle region, the growth factor domain region or the C-terminal region of the amino terminal fragment of urokinase or combinations thereof. The antibodies and/or conjugates thereof, which can include a therapeutic agent or a diagnostic agent may be used to treat, prevent or detect diseases such as, for example, cancer.

The antibodies may be an immunoglobulin of any class, e.g., IgG, IgM, IgA, IgD, IgE, etc. or of any isotype. In some embodiments, the antibodies are IgG1 antibodies. In other embodiments, the antibodies are IgG1 antibodies of the κ isotype. In still other embodiments, the antibodies are polyclonal, preferably affinity purified from a human or appropriate animal. In still other embodiments, the antibodies are monoclonal. In still other embodiments, the antibodies are monoclonal IgG1 antibodies. In still other embodiments, the antibodies are monoclonal IgG1 antibodies of the κ isotype. Polyclonal and monoclonal antibodies against the amino terminal fragment of urokinase may be prepared by conventional methods known to those of skill in the art.

The use of functionally active fragments of antibodies is also contemplated. Functionally active fragments of antibodies retain the ability to immunospecifically bind antigen as determined by any method known to those of skill in the art. Examples of functionally active fragments include, but are not limited to, fragments such as Fab, F(ab)₂, Fab′, F(ab′)₂ and Fᵥ derivatives which may readily prepared by methods known to the skilled artisan.

Single chain antibodies may also be used and can be prepared by methods known in the art (Ladner et al., U.S. Pat. No. 4,946,778, Bird, Science 1988, 242, 423-426; Huston et al., Proc. Natl. Acad. Sci. 1988, 85, 5879-5883; Ward et al., Nature 1988, 334, 544-546). The use of heavy chain and light chain dimers and diabodies is also contemplated.

Chimeric antibodies (i.e., where different portions of the antibody molecule are derived from different species) such as those having a variable region from derived from a murine antibody and a constant region derived from a human immunoglobulin (i.e., humanized antibodies) may also be used. Methods are known in the art for preparing chimeric and humanized antibodies (e.g., Neuberger et al., International Patent Application No. PCT/GB85/00392).

The antibodies and conjugates thereof will generally bind to the amino terminal fragment of urokinase (see SEQ ID NO 1 for the sequence of mature urokinase). In some embodiments, the amino terminal fragment of urokinase comprises amino acids 1-143 of SEQ ID NO 1. Antibodies which bind specifically to various parts of the amino terminal fragment such as the growth factor domain, the Kringle domain and the C-terminal domain are also within the scope of the present invention. In some embodiments, the growth factor domain comprises amino acids 1-48 of SEQ ID NO 1. In other embodiments, the Kringle domain comprises amino acids 49-135 of SEQ ID NO 1. In still other embodiments, the C-terminal domain comprises amino acids 136-143 of SEQ ID NO 1.

### 5.3 Antibody Conjugates

Antibodies may be modified by the covalent attachment of any type of molecule as long as the modification does not prevent or inhibit immunospecific binding to the amino terminal fragment of urokinase. For example, antibodies may be modified by glycosylation, acetylation, pegylation, phosphorylation, amidation, proteolytic cleavage, linkage to cellular ligand or protein, etc. In some embodiments, antibodies are conjugated to a therapeutic agent or a diagnostic agent either directly or through a linking moiety.

In some embodiments, the linking moiety is first attached to a diagnostic or therapeutic agent to form a linking moiety intermediate which is then further attached to an antibody. In other embodiments, the linking moiety can also be first attached to the antibody to form a linking moiety antibody intermediate which can then be attached to a diagnostic agent or therapeutic agent.

Typically, a linking moiety includes a linker and a linking group for conjugating a therapeutic agent or diagnostic agent to an antibody. The nature of the linker will depend upon the particular application and the type of conjugation desired as the linker may be hydrophilic or hydrophobic, long or short, rigid or flexible. The linker may be optionally substituted with one ore more linking groups which may be either the same or different, accordingly providing polyvalent linking moieties which are capable of conjugating multiple therapeutic agents or diagnostic agents with an antibody.

A wide variety of linkers comprised of stable bonds suitable for spacing linking groups from the antibody are known in the art, and include by way of example and not limitation, alkyl, heteroalkyl, acyclic heteroatomic bridges, aryl, aryl-aryl, arylalkyl, heteroaryl, heteroaryl-heteroaryl, substituted heteroaryl-heteroaryl, heteroarylalkyl, heteroaryl-heteroalkyl and the like and their substituted analogs. Thus, the linker may include single, double, triple or aromatic carbon-carbon bonds, nitrogen-nitrogen bonds, carbon-nitrogen, carbon-oxygen bonds and/or carbon-sulfur bonds. Accordingly, functionalities such as carbonyls, ethers, thioethers. carboxamides, sulfonamides, ureas, urethanes, hydrazines, etc. may be included in a linker.

Choosing a suitable linker is within the capabilities of those of skill in the art. For example, where a rigid linker is desired, the linker may be rigid polyunsaturated alkyl or an aryl, biaryl, heteroaryl, etc. Where a flexible linker is desired, the linker may be a flexible peptide such as Gly-Gly-Gly or a flexible saturated alkanyl or heteroalkanyl. Hydrophilic linkers may be, for example, polyalcohols or polyethers such as polyalkyleneglycols. Hydrophobic linkers may be, for example, alkyls or aryls.

Preferably, a linking group is capable of mediating formation of a covalent bond with a complementary reactive functionality of, for example, the antibody to provide the therapeutic agent or diagnostic agent conjugated to the antibody. Accordingly, the linking group may be any reactive functional group known to those of skill in the art that will react with common chemical groups found in antibodies (e.g., amino, sulflhydryl, hydroxyl, carboxylate, imidizaloyl, guandinium, amide, etc.). The linking group may be, for example, a photochemically activated group, an electrochemically activated group, a free radical donor, a free radical acceptor, a nucleophilic group or an electrophilic group. However, those of skill in the art will recognize that a variety of functional groups which are typically unreactive under certain reaction conditions can be activated to become reactive. Groups that can be activated to become reactive include, e.g., alcohols, carboxylic acids and esters, including salts thereof.

The linking group may be, for example, —NHR , —NH₂, —OH, —SH, halogen, —CHO, —R¹CO, —SO₂H, —PO₂H, —N₃, —CN, —CO₂H, —SO₃H, —PO₃H, —PO₂(O R¹)H, —CO₂R¹, —SO₃R¹ or —PO(OR¹)₂ where R¹ is alkyl. Preferably, the linking group is —NHR¹, —NH₂, —OH, —SH, —CHO, —CO₂H, R¹CO—, halogen and —CO₂R¹.

Some embodiments of the linker and the linking group include, for example, compounds where the linker is —(CH₂)ₙ—, n is an integer between 1 and 8, the linking group is —NH₂, —OH, —CO₂H, and —CO₂R¹ and the corresponding analogues where any suitable hydrogen is substituted. Other embodiments of the linking moiety include any amino acid, which may be, for example, a D or L amino acid. Thus, the linking moiety may be a dipeptide, a tripeptide or a tetrapeptide comprised of any combination of amino acids. The polarity of the peptide bond in these peptides may be either C—N or N—C.

Therapeutic agents and diagnostic agents may be linked to antibodies directly using a variety of conventional reactions known to the skilled artisan (Garnett, Adv. Drug Delivery Rev. 2001, 53, 171-216; Meyer et al., Annual Reports in Medicinal Chemistry 2003, 38, 229-237; Trail et al., Cancer Immunol. Immunother. 2003, 52, 328-337). For example, condensation reagents (e.g., carbodiimides, carbonyldiimidazoles, etc.) may be used to form an amide bond linkage between an amino group of the therapeutic or diagnostic agent and the carboxylic acid groups of residues such as glutamic acid and aspartic acid. Alternatively, the carbohydrate residues of antibodies may be linked to therapeutic or diagnostic agents through Schiff base formation (e.g., Sivan et al., U.S. Pat. No. 5,521,290; Shih et al., U.S. Pat. No. 5,057,313) followed by in situ reduction.

Similar methods may be used to attach therapeutic agents and diagnostic agents containing a linker and linking group to antibodies. For example, diagnostic agents and therapeutic agents containing a linker and linking group may be attached to the amino group of lysine, the carboxylic acid groups of glutamic acid and aspartic acid, the sulfhydryl group of cysteine, the hydroxyl groups of threonine and serine and the various moieties of aromatic amino acids using conventional approaches known to the skilled artisan. In general, selection of an appropriate strategy for conjugating diagnostic agents or therapeutic agents to an antibody either directly or through a linker and linking group is well within the ambit of the skilled artisan.

Therapeutic agents which can be conjugated to antibodies and fragments thereof include, but are not limited to, radionuclides, protein toxins (e.g., ricin, Pseudomonas exotoxin, diptheria toxin, saporin, pokeweed antiviral protein, bouganin, etc.), cytotoxic cancer agents, camptothecins (e.g., 9-nitrocamptothecin (9NC), 9-aminocamptothecin (9AC), 10-aminocamptothecin, 9-chlorocamptothecin, 10,11-methylendioxycamptothecin, irinothecin, aromatic camptothecin esters, alkyl camptothecin esters, topotecan, (1S,9S)-1-amino-9-ethyl-5-fluoro-2,3-dihydro-9-hydroxy-4-methyl-1H, 12H-benzo[de]pyrano[3′,4′:6,7]indolizino[1,2-b]quinoline-10,13(9H, 15H)-dione methanesulfonate dihydrate (DX-8951f), 7-[(2-trimethylsilyl)ethyl]-20(S)camptothecin (BNP1350), Rubitecan, Exatecan, Lurtotecan, Diflomotecan and other homocamptothecins, etc.), taxanes (e.g., taxol), epithilones, calicheamycins, hydroxy urea, cytarabine, cyclophosamide, ifosamide, nitrosureas, cisplatin, mitomycins maytansines, carboplatin, dacarbazine, procarbazine, etoposides, tenoposide, bleomycin, doxurobicin, 2-pyrrolinodoxurobicin. daunomycin, idarubican, daunorubicin, dactinomycin, plicamycin, mitoxantrone, asparginase, dihydroxy anthracine dione, mithrimycin, actinomycin D, 1-dehydrotestosterone, cytochlasins, vinblastine, vincristine, vinorelbine, paclitaxel, docetaxel, gramicidin D, glucocorticoids, anthracyclines, procaine, teracaine, lidocaine, propanolol, puromycin, methotrexate, 6-mercaptopurine, 6-thioguanine, mustard toxins, anthyrimycin, paclitaxel, alkylating agents (e.g., mechoremethamine, thioepa chlorambucil, melphalan, carmustine, loustine, cyclothosphamide, busulfan, dibromomannitol, streptozotocin, etc.) homologues and analogues thereof. Preferably, the therapeutic agent is a cytotoxic cancer agent, such as, for example, a taxane, a camptothecin, an epithilone or an anthracycline. In some embodiments, the therapeutic agent is doxorubicin. In other embodiments, the therapeutic agent is a radionuclide. In still other embodiments, the therapeutic agent is a camptothecin.

The term “diagnostically labeled” means that an antibody has an attached diagnostically detectable label. Many different labels exist in the art and methods of labeling are well known the skilled artisan. General classes of labels, which can be used in the present invention, include but are not limited to, radioactive isotopes, paramagnetic isotopes, compounds which can be imaged by positron emission tomography (PET), fluorescent or colored compounds, compounds which can be imaged by magnetic resonance, chemiluminescent compounds, bioluminescent compounds, etc. Suitable detectable labels include, but are not limited to, radioactive, fluorescent, fluorogenic or chromogenic labels. Useful radiolabels (radionuclides), which are detected simply by gamma counter, scintillation counter or autoradiography include, but are not limited to, ³H, ¹²⁵I, ¹³¹I, ³⁵S and ¹⁴C.

Methods and compositions for complexing metals to larger molecules, such as antibodies are well known in the art. The metals include detectable metal atoms, such as radionuclides, and may be complexed to antibodies and conjugates thereof by conventional methods (See, e.g., U.S. Pat. Nos. 5,627,286, 5,618,513, 5,567,408, 5,443,816 and 5,561,220).

Common fluorescent labels include, but are not limited to, fluorescein, rhodamine, dansyl, phycoerythrin, phycocyanin, allophycocyanin, o-phthaldehyde and fluorescamine (Haugland, Handbook of Fluorescent Probes and Research Chemicals, Sixth Ed., Molecular Probes, Eugene, Oreg., 1996) may be used to label antibodies and/or conjugates thereof. Fluorescein, fluorescein derivatives and fluorescein-like molecules such as Oregon Green™ and its derivatives, Rhodamine Green™ and Rhodol Green™, may be coupled to amine groups using, for example, the isothiocyanate, succinimidyl ester or dichlorotriazinyl-reactive groups. Similarly, fluorophores may also be coupled to thiols using maleimide, iodoacetamide, and aziridine-reactive groups. In some embodiments, the fluorophore is a long wavelength rhodamines, such as Rhodamine Green™ derivatives with substituents on the nitrogens. This group includes the tetramethylrhodamines, X-rhodamines and Texas Red™ derivatives. In other embodiments, the fluorophore is one excited by ultraviolet light. Examples include but are not limited to, cascade blue, coumarin derivatives, naphthalenes (of which dansyl chloride is a member), pyrenes and pyridyloxazole derivatives.

Inorganic materials such as semiconductor nanocrystals (Bruchez, et al., 1998, Science 281:2013-2016) and quantum dots, e.g., zinc-sulfide-capped Cd selenide (Chan, et al., Science 1998, 281:2016-2018) may also be used as diagnostic labels.

Antibodies and/or conjugates thereof can also be labeled with fluorescence-emitting metals such as ¹⁵²Eu or others of the lanthanide series. These metals can be attached to antibodies and/or conjugates thereof through acyl chelating groups such as diethylenetriaminepentaacetic acid (DTPA), ethylene-diamine-tetraacetic acid (EDTA), etc.

Radionuclides may be attached to antibodies and/or conjugates thereof either directly or indirectly using an acyl chelating group such as DTPA and EDTA for in vivo diagnosis. The chemistry of chelation is well known in the art and varying ranges of chelating agent to antibody may be used to provide the labeled antibody. Of course, the labeled antibody must retain the ability to bind the amino terminal fragment of urokinase.

Any radionuclide having diagnostic or therapeutic value can be used as the radiolabel in the present invention. In some embodiments, the radionuclide is a γ-emitting or beta-emitting radionuclide, for example, one selected from the lanthanide or actinide series of the elements. Positron-emitting radionuclides, e.g. ⁶⁸Ga or ⁶⁴Cu, may also be used. Suitable gamma-emitting radionuclides include those which are useful in diagnostic imaging applications. The gamma -emitting radionuclides preferably have a half-life of from 1 hour to 40 days, preferably from 12 hours to 3 days. Examples of suitable gamma -emitting radionuclides include ⁶⁷Ga, ¹¹¹In, ^{99m}Tc, ¹⁶⁹Yb and ¹⁸⁶Re. In some embodiments, the radionuclide is ^{99m}Tc. Examples of useful radionuclides (ordered by atomic number) are ⁶⁷Cu, ⁶⁷Ga, ⁶⁸Ga, ⁷²As, ⁸⁹Zr, ⁹⁰Y, ⁹⁷Ru, ⁹⁹Tc, ¹¹¹In, ¹²³I, ¹³¹I, ¹⁶⁹Yb, ¹⁸⁶Re, and ²⁰¹Tl. Though limited work have been done with positron-emitting radiometals as labels, certain proteins, such as transferrin and human serum albumin, have been labeled with ⁶⁸Ga.

Metals (not radioisotopes) useful for magnetic resonance imaging include gadolinium, manganese, copper, iron, gold and europium. In some embodiments, the metal is gadolinium. Generally, the amount of labeled antibody needed for detectability in diagnostic use will vary depending on considerations such as age, condition, sex, and extent of disease in the patient, contraindications, if any, and other variables, and is to be adjusted by the individual physician or diagnostician. Dosage can vary from 0.01 mg/kg to 100 mg/kg.

Antibodies and/or conjugates thereof may also be detected by coupling to a phosphorescent or a chemiluminescent compound, as is well known to the skilled artisan. Chemiluminescent compounds include but are not limited to, luminol, isoluminol, theromatic acridinium ester, imidazole, acridinium salt and oxalate ester. Similarly, bioluminescent compounds may be used to detect antibodies and/or conjugates thereof and include, but are not limited to, luciferin, luciferase and aequorin.

Colorimetric detection, based on chromogenic compounds which have, or result in, chromophores with high extinction coefficients may also be used to detect antibodies.

The use of antibodies which are genetically fused to a protein toxin is contemplated herein (Frankel et al., Sem. Oncol. 2003, 30, 545-557; Kreitman, Curr. Opin. Molec. Therapeutics 2003, 5, 44-51; Kreitman, Curr. Opin. Invest. Drugs 2001, 2, 1282-1293). Protein toxins include, but are not limited to, ricin, Pseudomonas exotoxin, diptheria toxin, saporin, pokeweed antiviral protein, bouganin, analogues and homologues thereof. Preferred protein toxins are Pseudomonas exotoxin and diptheria toxin. Antibodies fused to protein toxins can be made by recombinant DNA methods described in Section 5.5, infra (See e.g., Brinkman et al, Proc. Natl. Acad. Sci. 1991, 88, 8616-8620; Haggerty et al., Toxicol. Pathol. 1999, 87-94; Damis et al., J. Pharm. Pharmacol. 2000, 52, 671-678).

### 5.4 Assays

Those of skill in the art will appreciate that the in vitro and in vivo assays useful for measuring the activity of antibodies and conjugates thereof described herein are illustrative rather than comprehensive.

### 5.4.1 Assay for Endothelial Cell Migration

For endothelial cell (EC) migration, transwells are coated with type I collagen (50 μg/mL) by adding 200 μL of the collagen solution per transwell, then incubating overnight at 37° C. The transwells are assembled in a 24-well plate and a chemoattractant (e.g., FGF-2) is added to the bottom chamber in a total volume of 0.8 mL media. ECs, such as human umbilical vein endothelial cells (HUVEC), which have been detached from monolayer culture using trypsin, are diluted to a final concentration of about 10⁶ cells/mL with serum-free media and 0.2 mL of this cell suspension is added to the upper chamber of each transwell. Inhibitors to be tested may be added to both the upper and lower chambers and the migration is allowed to proceed for 5 hrs in a humidified atmosphere at 37° C. The transwells are removed from the plate stained using DiffQuik®. Cells which did not migrate are removed from the upper chamber by scraping with a cotton swab and the membranes are detached, mounted on slides, and counted under a high-power field (400×) to determine the number of cells migrated.

### 5.4.2 Biological Assay of Anti-Invasive Activity

The ability of cells such as ECs or tumor cells (e.g., PC-3 human prostatic carcinoma) cells to invade through a reconstituted basement membrane (Matrigel®) in an assay known as a Matrigel® invasion assay system has been described in detail in the art (Kleinman et al., Biochemistry 1986, 25: 312-318; Parish et al., 1992, Int. J. Cancer 52:378-383). Matrigel® is a reconstituted basement membrane containing type IV collagen, laminin, heparan sulfate proteoglycans such as perlecan, which bind to and localize bFGF, vitronectin as well as transforming growth factor-β (TGFβ). urokinase-type plasminogen activator (uPA), tissue plasminogen activator (tPA) and the serpin known as plasminogen activator inhibitor type 1 (PAI-1) (Chambers et al., Canc. Res. 1995. 55:1578-1585). It is accepted in the art that results obtained in this assay for antibodies and/or conjugates thereof which target extracellular receptors or enzymes are predictive of the efficacy of these antibodies and/or conjugates thereof in vivo (Rabbani et al., Int. J. Cancer 1995, 63: 840-845).

Such assays employ transwell tissue culture inserts. Invasive cells are defined as cells which are able to traverse through the Matrigel® and upper aspect of a polycarbonate membrane and adhere to the bottom of the membrane. Transwells (Costar) containing polycarbonate membranes (8.0 μm pore size) are coated with Matrigel® (Collaborative Research), which has been diluted in sterile PBS to a final concentration of 75 μg/mL (60 μL of diluted Matrigel® per insert), and placed in the wells of a 24-well plate. The membranes are dried overnight in a biological safety cabinet, then rehydrated by adding 100 μL of DMEM containing antibiotics for 1 hour on a shaker table. The DMEM is removed from each insert by aspiration and 0.8 mL of DMEM/10% FBS/antibiotics is added to each well of the 24-well plate such that it surrounds the outside of the transwell (“lower chamber”). Fresh DMEM/antibiotics (100 μL), human Glu-plasminogen (5 μg/mL), and any inhibitors to be tested are added to the top, inside of the transwell (“upper chamber”). The cells which are to be tested are trypsinized and resuspended in DMEM/antibiotics, then added to the top chamber of the transwell at a final concentration of 800,000 cells/mL. The final volume of the upper chamber is adjusted to 200 μL. The assembled plate is then incubated in a humid 5% CO₂ atmosphere for 72 hours. After incubation, the cells are fixed and stained using DiffQuik® (Giemsa stain) and the upper chamber is then scraped using a cotton swab to remove the Matrigel® and any cells which did not invade through the membrane. The membranes are detached from the transwell using an X-acto® blade, mounted on slides using Permount® and cover-slips, then counted under a high-powered (400×) field. An average of the cells invaded is determined from 5-10 fields counted and plotted as a function of inhibitor concentration.

### 5.4.3 Tube-Formation Assays of Anti-Angiogenic Activity

Endothelial cells, for example, human umbilical vein endothelial cells (HUVEC) or human microvascular endothelial cells (HMVEC) which can be prepared or obtained commercially, are mixed at a concentration of 2×10⁵ cells/mL with fibrinogen (5 mg/mL in phosphate buffered saline (PBS) in a 1:1 (v/v) ratio. Thrombin is added (5 units/mL final concentration) and the mixture is immediately transferred to a 24-well plate (0.5 mL per well). The fibrin gel is allowed to form and then VEGF and bFGF are added to the wells (each at 5 ng/mL final concentration) along with the test compound. The cells are incubated at 37° C. in 5% CO₂ for 4 days at which time the cells in each well are counted and classified as either rounded, elongated with no branches, elongated with one branch, or elongated with 2 or more branches. Results are expressed as the average of 5 different wells for each concentration of compound. Typically, in the presence of angiogenic inhibitors, cells remain either rounded or form undifferentiated tubes (e.g. 0 or 1 branch). This assay is recognized in the art to be predictive of angiogenic (or anti-angiogenic) efficacy in vivo (Min et al., Cancer Res. 1996, 56: 2428-2433).

In an alternate assay, endothelial cell tube formation is observed when endothelial cells are cultured on Matrigel® (Schnaper et al., J. Cell. Physiol. 1995, 165:107-118). Endothelial cells (1×10⁴ cells/well) are transferred onto Matrigel®-coated 24-well plates and tube formation is quantitated after 48 hrs. Inhibitors are tested by adding them either at the same time as the endothelial cells or at various time points thereafter. Tube formation can also be stimulated by adding (a) angiogenic growth factors such as bFGF or VEGF, (b) differentiation stimulating agents (e.g., PMA) or (c) a combination of these.

While not wishing to be bound by theory, this assay models angiogenesis by presenting to the endothelial cells a particular type of basement membrane, namely the layer of matrix which migrating and differentiating endothelial cells might be expected to first encounter. In addition to bound growth factors, the matrix components found in Matrigel® (and in basement membranes in situ) or proteolytic products thereof may also be stimulatory for endothelial cell tube formation which makes this model complementary to the fibrin gel angiogenesis model previously described (Blood et al., Biochim. Biophys. Acta 1990, 1032:89-118; Odedrat al., Pharmac. Ther. 1991, 49:111-124).

### 5.4.4 Assays for Inhibition of Proliferation

The ability of the antibodies and/or conjugates thereof to inhibit the proliferation of EC's may be determined in a 96-well format. Type I collagen (gelatin) is used to coat the wells of the plate (0.1-1 mg/mL in PBS, 0.1 mL per well for 30 minutes at room temperature). After washing the plate (3× w/PBS), 3-6,000 cells are plated per well and allowed to attach for 4 hrs (37° C./5% CO₂) in Endothelial Growth Medium (EGM; Clonetics ) or M199 media containing 0.1-2% FBS. The media and any unattached cells are removed at the end of 4 hrs and fresh media containing bFGF (1-10 ng/mL) or VEGF (1-10 ng/mL) is added to each well. Antibodies and/or conjugates thereof to be tested are added last and the plate is allowed to incubate (37° C./5% CO₂) for 24-48 hrs. MTS (Promega) is added to each well and allowed to incubate from 1-4 hrs. The absorbance at 490 nm, which is proportional to the cell number, is then measured to determine the differences in proliferation between control wells and those containing test antibodies and/or conjugates thereof.

A similar assay system can be set up with cultured adherent tumor cells. However, collagen may be omitted in this format. Tumor cells (e.g., 3,000-10,000/well) are plated and allowed to attach overnight. Serum free medium is then added to the wells, and the cells are synchronized for 24 hrs. Medium containing 10% FBS is then added to each well to stimulate proliferation. Antibodies and/or conjugates thereof to be tested are included in some of the wells. After 24 hrs, MTS is added to the plate and the assay developed and read as described above.

### 5.4.5 Assays of Cytotoxicity

The anti-proliferative and cytotoxic effects of antibodies and/or conjugates thereof may be determined for various cell types including tumor cells, ECs, fibroblasts and macrophages. This is especially useful when testing a antibody which has been conjugated to a therapeutic moiety such as a radiotherapeutic or a toxin. For example, a conjugate of one of the antibodies of the invention with Bolton-Hunter reagent which has been iodinated with ¹³¹I would be expected to inhibit the proliferation of cells expressing uPAR (most likely by inducing apoptosis). Anti-proliferative effects would be expected against tumor cells and stimulated endothelial cells but, under some circumstances not quiescent endothelial cells or normal human dermal fibroblasts. Any anti-proliferative or cytotoxic effects observed in the normal cells may represent non-specific toxicity of the conjugate.

A typical assay would involve plating cells at a density of 5-10,000 cells per well in a 96-well plate. The compound to be tested is added at a concentration 10× the IC₅₀ measured in a binding assay (this will vary depending on the conjugate) and allowed to incubate with the cells for 30 minutes. The cells are washed 3× with media, then fresh media containing [³H]thymidine (1 μCi/mL) is added to the cells and they are allowed to incubate at 37° C. in 5% CO₂ for 24 and 48 hours. Cells are lysed at the various time points using 1 M NaOH and counts per well determined using a β-counter. Proliferation may be measured non-radioactively using MTS reagent or CyQuant® to measure total cell number. For cytotoxicity assays (measuring cell lysis), a Promega 96-well cytotoxicity kit is used. If there is evidence of anti-proliferative activity, induction of apoptosis may be measured using TumorTACS (Genzyme).

### 5.4.6 Caspase-3 Activity

The ability of the antibodies and/or conjugates thereof to promote apoptosis of EC's may be determined by measuring activation of caspase-3. Type I collagen (gelatin) is used to coat a P100 plate and 5×10⁵ ECs are seeded in EGM containing 10% FBS. After 24 hours (at 37° C. in 5% CO₂) the medium is replaced by EGM containing 2% FBS, 10 ng/ml bFGF and the desired test compound. The cells are harvested after 6 hours, cell lysates prepared in 1% Triton and assayed using the EnzChek®Caspase-3 Assay Kit #1 (Molecular Probes) according to the manufactures' instructions.

### 5.4.7 Corneal Angiogenesis Model

The protocol used is essentially identical to that described by Volpert et al., J. Clin. Invest. 1996, 98:671-679. Briefly, female Fischer rats (120-140 gms) are anesthetized and pellets (5 μl) comprised of Hydron®, bFGF (150 nM), and the antibodies and/or conjugates thereof to be tested are implanted into tiny incisions made in the cornea 1.0-1.5 mm from the limbus. Neovascularization is assessed at 5 and 7 days after implantation. On day 7, animals are anesthetized and infused with a dye such as colloidal carbon to stain the vessels. The animals are then euthanized, the corneas fixed with formalin, and the corneas flattened and photographed to assess the degree of neovascularization. Neovessels may be quantitated by imaging the total vessel area or length or simply by counting vessels.

### 5.4.8 Matrigel® Plug Assay

This assay is performed essentially as described by Passaniti et al., 1992, Lab Invest. 67:519-528. Ice-cold Matrigel® (e.g., 500 μL) (Collaborative Biomedical Products, Inc., Bedford, Mass.) is mixed with heparin (e.g., 50 μg/ml), FGF-2 (e.g., 400 ng/ml) and the compound to be tested. In some assays, bFGF may be substituted with tumor cells as the angiogenic stimulus. The Matrigel® mixture is injected subcutaneously into 4-8 week-old athymic nude mice at sites near the abdominal midline, preferably 3 injections per mouse. The injected Matrigel® forms a palpable solid gel. Injection sites are chosen such that each animal receives a positive control plug (such as FGF-2+heparin), a negative control plug (e.g., buffer+heparin) and a plug that includes the compound being tested for its effect on angiogenesis, e.g., (FGF-2+heparin+compound). All treatments are preferably run in triplicate. Animals are sacrificed by cervical dislocation at about 7 days post injection or another time that may be optimal for observing angiogenesis. The mouse skin is detached along the abdominal midline, and the Matrigel® plugs are recovered and scanned immediately at high resolution. Plugs are then dispersed in water and incubated at 37° C. overnight. Hemoglobin (Hb) levels are determined using Drabkin's solution (e.g., obtained from Sigma) according to the manufacturers' instructions. The amount of Hb in the plug is an indirect measure of angiogenesis as it reflects the amount of blood in the sample. In addition, or alternatively, animals may be injected prior to sacrifice with a 0.1 ml buffer (preferably PBS) containing a high molecular weight dextran to which is conjugated a fluorophore. The amount of fluorescence in the dispersed plug, determined fluorimetrically, also serves as a measure of angiogenesis in the plug. Staining with mAb anti-CD31 (CD31 is “platelet-endothelial cell adhesion molecule or PECAM”) may also be used to confirm neovessel formation and microvessel density in the plugs.

### 5.4.9 Chick Chorioallantoic Membrane (CAM) Angiogenesis Assay

This assay is performed essentially as described by Nguyen et al., Microvascular Res. 1994, 47:31-40. A mesh containing either angiogenic factors (bFGF) or tumor cells plus inhibitors is placed onto the CAM of an 8-day old chick embryo and the CAM observed for 3-9 days after implantation of the sample. Angiogenesis is quantitated by determining the percentage of squares in the mesh which contain blood vessels.

### 5.4.10 In Vivo Assessment of Angiogenesis Inhibition and Anti-Tumor Effects Using the Matrigel® Plug Assay with Tumor Cells

In this assay, tumor cells, for example 1-5×10⁶ cells of the 3LL Lewis lung carcinoma or the rat prostate cell line MatLyLu, are mixed with Matrigel® and then injected into the flank of a mouse following the protocol described in Sec. B., above. A mass of tumor cells and a powerful angiogenic response can be observed in the plugs after about 5 to 7 days. The anti-tumor and anti-angiogenic action of a compound in an actual tumor environment can be evaluated by including it in the plug. Measurement is then made of tumor weight, Hb levels or fluorescence levels (of a dextran-fluorophore conjugate injected prior to sacrifice). To measure Hb or fluorescence, the plugs are first homogenized with a tissue homogenizer.

### 5.4.11 Xenograft Model of Subcutaneous (s.c.) Tumor Growth

Nude mice are inoculated with MDA-MB-231 cells (human breast carcinoma) and Matrigel® (1×10⁶ cells in 0.2 mL) s.c. in the right flank of the animals. The tumors are staged to 200 mm³ and then treatment with a test composition is initiated (100 μg/animal/day given q.d. IP). Tumor volumes are obtained every other day and the animals are sacrificed after 2 weeks of treatment. The tumors are excised, weighed and paraffin embedded. Histological sections of the tumors are analyzed by H and E, anti-CD31, Ki-67, TUNEL, and CD68 staining.

### 5.4.12 Xenograft Model of Metastasis

The antibodies and/or conjugates thereof are also tested for inhibition of late metastasis using an experimental metastasis model (Crowley et al., Proc. Natl. Acad. Sci. USA 1993, 90 5021-5025). Late metastasis involves the steps of attachment and extravasation of tumor cells, local invasion, seeding, proliferation and angiogenesis. Human prostatic carcinoma cells (PC-3) transfected with a reporter gene, preferably the green fluorescent protein (GFP) gene, but as an alternative with a gene encoding the enzymes chloramphenicol acetyl-transferase (CAT), luciferase or LacZ, are inoculated into nude mice. This approach permits utilization of either of these markers (fluorescence detection of GFP or histochemical colorimetric detection of enzymatic activity) to follow the fate of these cells. Cells are injected, preferably iv, and metastases identified after about 14 days, particularly in the lungs but also in regional lymph nodes, femurs and brain. This mimics the organ tropism of naturally occurring metastases in human prostate cancer. For example, GFP-expressing PC-3 cells (1×10⁶ cells per mouse) are injected iv into the tail veins of nude (nu/nu) mice. Animals are treated with a test composition at 100 μg/animal/day given q.d. IP. Single metastatic cells and foci are visualized and quantitated by fluorescence microscopy or light microscopic histochemistry or by grinding the tissue and quantitative colorimetric assay of the detectable label.

### 5.4.13 Inhibition of Spontaneous Metastasis In Vivo

The rat syngeneic breast cancer system employs Mat BIII rat breast cancer cells (Xing et al., Int. J. Cancer 1996, 67:423-429). Tumor cells, for example, about 10⁶ suspended in 0.1 mL PBS, are inoculated into the mammary fat pads of female Fisher rats. At the time of inoculation, a 14-day Alza osmotic mini-pump is implanted intraperitoneally to dispense the test antibody and/or conjugate thereof. The antibody and/or conjugate thereof (in PBS), is sterile filtered and placed in the minipump to achieve a release rate of about 4 mg/kg/day. Control animals receive vehicle (PBS) alone or a vehicle control peptide in the minipump. Animals are sacrificed at about day 14. In the rats treated with the antibodies and/or conjugates thereof, significant reductions in the size of the primary tumor and in the number of metastases in the spleen, lungs, liver, kidney and lymph nodes (enumerated as discrete foci) may be observed. Histological and immunohistochemical analysis reveal increased necrosis and signs of apoptosis in tumors in treated animals. Large necrotic areas are seen in tumor regions lacking neovascularization. Antibodies and/or conjugates thereof to which ¹³¹I is conjugated (either 1 or 2 I atoms per molecule of antibody) are effective radiotherapeutics and are found to be at least two-fold more potent than the unconjugated antibodies. In contrast, treatment with control antibodies fails to cause a significant change in tumor size or metastasis.

### 5.4.14 3LL Lewis Lung Carcinoma: Primary Tumor Growth

This tumor line arose spontaneously as carcinoma of the lung in a C57BL/6 mouse (Malave et al., J. Nat'l. Canc. Inst. 1979, 62:83-88). It is propagated by passage in C57BL/6 mice by subcutaneous (sc) inoculation and is tested in semiallogeneic C57BL/6×DBA/2 F₁ mice or in allogeneic C3H mice. Typically six animals per group for subcutaneously (sc) implant, or ten for intramuscular (im) implant are used. Tumor may be implanted sc as a 2-4 mm fragment, or im or sc as an inoculum of suspended cells of about 0.5-2×10⁶-cells. Treatment begins 24 hours after implant or is delayed until a tumor of specified size (usually approximately 400 mg) can be palpated. The test compound is administered ip daily for 11 days Animals are followed by weighing, palpation, and measurement of tumor size. Typical tumor weight in untreated control recipients on day 12 after im inoculation is 500-2500 mg. Typical median survival time is 18-28 days. A positive control compound, for example cyclophosphamide at 20 mg/kg/injection per day on days 1-11 is used. Results computed include mean animal weight, tumor size, tumor weight, survival time. For confirmed therapeutic activity, the test composition should be tested in two multi-dose assays.

### 5.4.15 3LL Lewis Lung Carcinoma: Primary Growth and Metastasis Model

This assay is well known in the art (Gorelik et al., J. Nat'l. Canc. Inst. 1980, 65:1257-1264; Gorelik et al., Rec. Results Canc. Res. 1980, 75:20-28; Isakov et al., Invasion Metas. 2:12-32 (1982); Talmadge et al., J. Nat'l. Canc. Inst. 1982, 69:975-980; Hilgard et al., Br. J. Cancer 1977, 35:78-86). Test mice are male C57BL/6 mice, 2-3 months old. Following sc, im, or intra-footpad implantation, this tumor produces metastases, preferentially in the lungs. With some lines of the tumor, the primary tumor exerts anti-metastatic effects and must first be excised before study of the metastatic phase (see also U.S. Pat. No. 5,639,725). Single-cell suspensions are prepared from solid tumors by treating minced tumor tissue with a solution of 0.3% trypsin. Cells are washed 3 times with PBS (pH 7.4) and suspended in PBS. Viability of the 3LL cells prepared in this way is generally about 95-99% (by trypan blue dye exclusion). Viable tumor cells (3×10⁴-5×10⁶) suspended in 0.05 ml PBS are injected subcutaneously, either in the dorsal region or into one hind foot pad of C57BL/6 mice. Visible tumors appear after 3-4 days after dorsal sc injection of 10⁶ cells. The day of tumor appearance and the diameters of established tumors are measured by caliper every two days. The treatment is given as one to five doses of peptide or derivative, per week. In another embodiment, the peptide is delivered by osmotic minipump.

In experiments involving tumor excision of dorsal tumors, when tumors reach about 1500 mm³ in size, mice are randomized into two groups: (1) primary tumor is completely excised; or (2) sham surgery is performed and the tumor is left intact. Although tumors from 500-3000 mm³ inhibit growth of metastases, 1500 mm³ is the largest size primary tumor that can be safely resected with high survival and without local regrowth. After 21 days, all mice are sacrificed and autopsied.

Lungs are removed and weighed. Lungs are fixed in Bouin's solution and the number of visible metastases is recorded. The diameters of the metastases are also measured using a binocular stereoscope equipped with a micrometer-containing ocular under 8× magnification. On the basis of the recorded diameters, it is possible to calculate the volume of each metastasis. To determine the total volume of metastases per lung, the mean number of visible metastases is multiplied by the mean volume of metastases. To further determine metastatic growth, it is possible to measure incorporation of ¹²⁵IdUrd into lung cells (Thakur et al., J. Lab. Clin. Med. 1977, 89:217-228). Ten days following tumor amputation, 25 μg of fluorodeoxyuridine is inoculated into the peritoneums of tumor-bearing (and, if used, tumor-resected mice). After 30 min, mice are given 1 μCi of ¹²⁵IdUrd (iododeoxyuridine). One day later, lungs and spleens are removed and weighed, and a degree of ¹²⁵IdUrd incorporation is measured using a gamma counter.

In mice with footpad tumors, when tumors reach about 8-10 mm in diameter, mice are randomized into two groups: (1) legs with tumors are amputated after ligation above the knee joints; or (2) mice are left intact as nonamputated tumor-bearing controls. (Amputation of a tumor-free leg in a tumor-bearing mouse has no known effect on subsequent metastasis, ruling out possible effects of anesthesia, stress or surgery). Mice are killed 10-14 days after amputation. Metastases are evaluated as described above.

Statistics: Values representing the incidence of metastases and their growth in the lungs of tumor-bearing mice are not normally distributed. Therefore, non-parametric statistics such as the Mann-Whitney U-Test may be used for analysis. Study of this model by Gorelik et al. (1980, supra) showed that the size of the tumor cell inoculum determined the extent of metastatic growth. The rate of metastasis in the lungs of operated mice was different from primary tumor-bearing mice. Thus in the lungs of mice in which the primary tumor had been induced by inoculation of larger doses of 3LL cells (1-5×10⁶) followed by surgical removal, the number of metastases was lower than that in nonoperated tumor-bearing mice, though the volume of metastases was higher than in the nonoperated controls. Using ¹²⁵IdUrd incorporation as a measure of lung metastasis, no significant differences were found between the lungs of tumor-excised mice and tumor-bearing mice originally inoculated with 10⁶ 3LL cells. Amputation of tumors produced following inoculation of 10⁵ tumor cells dramatically accelerated metastatic growth. These results were in accord with the survival of mice after excision of local tumors. The phenomenon of acceleration of metastatic growth following excision of local tumors had been repeatedly observed (for example, see U.S. Pat. No. 5,639,725). These observations have implications for the prognosis of patients who undergo cancer surgery.

### 5.4.16 Assay for Antibody Binding to uPA on Whole Cells

The urokinase amino terminal fragment targeting antibody and/or conjugate thereof is readily tested for binding to uPA, preferably, by measuring inhibition of the binding of [¹²⁵I]DFP-uPA to uPAR in a competitive ligand-binding assay. The assay may employ whole cells that express uPAR, for example cells lines such as RKO or HeLa. A preferred assay is conducted as follows. Cells (about 5×10⁴/well) are plated in medium (e.g., MEM with Earle's salts/10% FBS+antibiotics) in 24-well plates, then incubated in a humid 5% CO₂ atmosphere until the cells reach 70% confluence. Catalytically inactivated high molecular weight uPA (DFP-uPA) is radioiodinated using lodo-gen® (Pierce) to a specific activity of about 250,000 cpm/mg. The cell-containing plates are then chilled on ice and the cells are washed twice (5 minutes each) with cold PBS/0.05% Tween-80. Test antibodies and/or conjugates thereof are serially diluted in cold PBS/0.1% BSA/0.01% Tween-80 and added to each well to a final volume of 0.3 mL 10 minutes prior to the addition of the [¹²⁵I]DFP-uPA. Each well then receives 9500 cpm of [¹²⁵I]DFP-uPA at a final concentration of 0.2 nM). The plates are then incubated at 4° C. for 2 hrs, after which time the cells are washed 3× (5 minutes each) with cold PBS/0.05% Tween-80. NaOH (1N) is added to each well in 0.5 mL to lyse the cells, and the plate is incubated for 5 minutes at room temperature or until all the cells in each well are lysed as determined by microscopic examination. The contents of each well are then aspirated and the total counts in each well determined using a gamma counter. Each compound is tested in triplicate and the results are expressed as a percentage of the total radioactivity measured in wells containing [¹²⁵I]DFP-uPA alone, which is taken to represent maximum (100%) binding.

The inhibition of binding of [¹²⁵I]DFP-uPA to uPAR is usually dose-related, such that the concentration of the test compound necessary to produce a 50% inhibition of binding (the IC₅₀ value), which is expected to fall in the linear part of the curve, is easily determined. In general, antibodies and/or conjugates thereof have IC₅₀ values of less than about 10⁻⁵ M. Preferably, antibodies and/or conjugates thereof have IC₅₀ values of less than about 10⁻⁶ M, more preferably, less than about 10⁻⁷M.

### 5.5 Recombinant DNA Methods

General methods of molecular biology have been amply described in the art (Sambrook, et al., Molecular Cloning: A Laboratory Manual, 2nd (or later) Edition, Cold Spring Harbor Press, Cold Spring Harbor, N.Y., 1989; Ausube et al., Current Protocols in Molecular Biology, Vol. 2, Wiley-Interscience, New York, (current edition); Kriegler, Gene Transfer and Expression: A Laboratory Manual (1990); Glover, D M, editor, DNA Cloning: A Practical Approach, vol. I & II, IRL Press, 1985; Alberts et al., Molecular Biology of the Cell, 2nd (or later) Ed., Garland Publishing, Inc., New York, N.Y. (1989); Watson et al., Recombinant DNA, 2nd (or later) Ed., Scientific American Books, New York, 1992; and Old et al., Principles of Gene Manipulation. An Introduction to Genetic Engineering, 2nd (or later) Ed., University of California Press, Berkeley, Calif. (1981)).

Unless otherwise indicated, a particular nucleic acid sequence is intended to encompasses conservative substitution variants thereof (e.g., degenerate codon substitutions) and a complementary sequence. The term “nucleic acid” is synonymous with “polynucleotide” and is intended to include a gene, a cDNA molecule, an mRNA molecule, as well as a fragment of any of these such as an oligonucleotide, and further, equivalents thereof (explained more fully below). Sizes of nucleic acids are stated either as kilobases (kb) or base pairs (bp). These are estimates derived from agarose or polyacrylamide gel electrophoresis (PAGE), from nucleic acid sequences which are determined by the user or published. Protein size is stated as molecular mass in kilodaltons (kDa) or as length (number of amino acid residues). Protein size is estimated from PAGE, from sequencing, from presumptive amino acid sequences based on the coding nucleic acid sequence or from published amino acid sequences.

Specifically, DNA molecules encoding the amino acid sequence corresponding to antibodies, or active variants thereof, can be synthesized by the polymerase chain reaction (PCR) (see, for example, U.S. Pat. No. 4,683,202) using primers derived the sequence of the protein disclosed herein. These cDNA sequences can then be assembled into a eukaryotic or prokaryotic expression vector and the resulting vector can be used to direct the synthesis of the fusion polypeptide or its fragment or derivative by appropriate host cells, for example COS or CHO cells.

Prokaryotic or eukaryotic host cells transformed or transfected to express antibodies and/or fragments thereof are within the scope of the invention. For example, the antibodies and/or fragments may be expressed in bacterial cells such as E. coli, insect cells (baculovirus), yeast, or mammalian cells such as Chinese hamster ovary cells (CHO) or human cells (which are preferred for human therapeutic use of the transfected cells). Other suitable hosts are known to those skilled in the art. Expression in eukaryotic cells leads to partial or complete glycosylation and/or formation of relevant inter- or intra-chain disulfide bonds of the recombinant polypeptide. Examples of vectors for expression in yeast S. cerevisiae include pYepSec1 (Baldari et al., 1987, EMBO J. 6:229-234), pMFa (Kurjan et al. 1982 Cell 30:933-943), pJRY88 (Schultz et al., 1987, Gene 54:113-123), and pYES2 (Invitrogen Corporation, San Diego, Calif.). Baculovirus vectors available for expression of proteins in cultured insect cells (SF 9 cells) include the pAc series (Smith et al., 1983, Mol. Cell Biol. 3:2156-2165) and the pVL series (Lucklow et al., (1989) Virology 170:31-39). Generally, COS cells (Gluzman 1981 Cell 23:175-182) are used in conjunction with such vectors as pCDM 8 (Aruffo et al., supra, for transient amplification/expression in mammalian cells, while CHO (dhfr-negative CHO) cells are used with vectors such as pMT2PC (Kaufman et al., 1987, EMBO J. 6:187-195) for stable amplification/expression in mammalian cells. The NS0 myeloma cell line (a glutamine synthetase expression system.) is available from Celltech Ltd.

Construction of suitable vectors containing the desired coding and control sequences employs standard ligation and restriction techniques which are well understood in the art. Isolated plasmids, DNA sequences, or synthesized oligonucleotides are cleaved, tailored, and re-ligated in the form desired. The DNA sequences which form the vectors are available from a number of sources. Backbone vectors and control systems are generally found on available “host” vectors which are used for the bulk of the sequences in construction. For the pertinent coding sequence, initial construction may be, and usually is, a matter of retrieving the appropriate sequences from cDNA or genomic DNA libraries. However, once the sequence is disclosed it is possible to synthesize the entire gene sequence in vitro starting from the individual nucleotide derivatives. The entire gene sequence for genes of sizeable length, e.g., 500-1000 bp may be prepared by synthesizing individual overlapping complementary oligonucleotides and filling in single stranded nonoverlapping portions using DNA polymerase in the presence of the deoxyribonucleotide triphosphates. This approach has been used successfully in the construction of several genes of known sequence. See, for example, Edge, Nature 1981, 292:756; Nambair et al., Science 1984, 223:1299; and Jay, J. Biol. Chem. 1984, 259:6311.

Synthetic oligonucleotides are prepared by either the phosphotriester method as described by references cited above or the phosphoramidite method as described by Beaucage et al., Tetrahedron Lett. 1981, 22:1859; and Matteucci et al., J. Am. Chem. Soc. 1981, 103:3185 and can be prepared using commercially available automated oligonucleotide synthesizers. Kinase treatment of single strands prior to annealing or for labeling is achieved using well-known methods.

Once the components of the desired vectors are thus available, they can be excised and ligated using standard restriction and ligation procedures. Site-specific DNA cleavage is performed by treating with the suitable restriction enzyme (or enzymes) under conditions which are generally understood in the art, and the particulars of which are specified by the manufacturer of these commercially available restriction enzymes. See, e.g., New England Biolabs, Product Catalog. If desired, size separation of the cleaved fragments may be performed by polyacrylamide gel or agarose gel electrophoresis using standard techniques. A general description of size separations is found in Meth. Enzymol. (1980) 65:499-560. Any of a number of methods are used to introduce mutations into the coding sequence to generate variants if these are to be produced recombinantly. These mutations include simple deletions or insertions, systematic deletions, insertions or substitutions of clusters of bases or substitutions of single bases. Modifications of the DNA sequence are created by site-directed mutagenesis, a well-known technique for which protocols and reagents are commercially available (Zoller et al., Nucleic Acids Res. 1982, 10:6487-6500 and Adelman et al., DNA 1983, 2:183-193)). The isolated DNA is analyzed by restriction and/or sequenced by the dideoxy nucleotide method of Sanger, Proc. Natl. Acad. Sci. USA 1977, 74:5463) as further described by Messing, et al., Nucleic Acids Res. 1981, 9:309, or by the method of Maxam et al., Meth. Enzymol., supra.

Vector DNA can be introduced into mammalian cells via conventional techniques such as calcium phosphate or calcium chloride co-precipitation, DEAE-dextran-mediated transfection, lipofection, or electroporation. Suitable methods for transforming host cells can be found in Sambrook et al. supra and other standard texts. In fusion expression vectors, a proteolytic cleavage site is introduced at the junction of the reporter group and the target protein to enable separation of the target protein from the reporter group subsequent to purification of the fusion protein. Proteolytic enzymes for such cleavage and their recognition sequences include Factor Xa, thrombin and enterokinase.

### 5.6 Therapeutic Uses

An antibody and/or conjugates thereof and/or a pharmaceutical composition thereof is administered to a patient, preferably a human, suffering from a disease characterized by cell migration, cell invasion or cell proliferation, angiogenesis or metastasis. Such diseases or conditions may include primary growth of solid tumors or leukemias and lymphomas, metastasis, invasion and/or growth of tumor metastases, benign hyperplasias, atherosclerosis, myocardial angiogenesis, angiofibroma, arteriovenous malformations, post-balloon angioplasty vascular restenosis, neointima formation following vascular trauma, vascular graft restenosis, coronary collateral formation, deep venous thrombosis, ischemic limb angiogenesis, telangiectasia, pyogenic granuloma, corneal diseases, rubeosis, neovascular glaucoma, diabetic and other retinopathy, retrolental fibroplasia, diabetic neovascularization, macular degeneration, endometriosis, arthritis, fibrosis associated with chronic inflammatory conditions including psoriasis scleroderma, hemangioma, hemophilic joints, hypertrophic scars, Osler-Weber syndrome, psoriasis, pyrogenic granuloma, retrolental fibroplasia, scleroderma, Von-Hippel-Landau syndrome, trachoma, vascular adhesions, lung fibrosis, chemotherapy-induced fibrosis, wound healing with scarring and fibrosis, peptic ulcers, fractures, keloids, and disorders of vasculogenesis, hematopoiesis, ovulation, menstruation, pregnancy and placentation, or any other disease or condition in which cell invasion or angiogenesis is pathogenic or undesired.

More recently, it has become apparent that angiogenesis inhibitors may play a role in preventing inflammatory angiogenesis and gliosis following traumatic spinal cord injury, thereby promoting the reestablishment of neuronal connectivity (Wamil et al., Proc. Natl. Acad. Sci. 1998, 95:13188-13193). Therefore, antibodies and/or conjugates thereof and/or pharmaceutical compositions are administered as soon as possible after traumatic spinal cord injury and for several days up to about two weeks thereafter to inhibit angiogenesis and gliosis that would sterically prevent reestablishment of neuronal connectivity. The treatment reduces the area of damage at the site of spinal cord injury and facilitates regeneration of neuronal function and thereby prevents paralysis. The antibodies and/or conjugates thereof are expected also to protect axons from Wallerian degeneration, reverse aminobutyrate-mediated depolarization (occurring in traumatized neurons), and improve recovery of neuronal conductivity of isolated central nervous system cells and tissue in culture.

Further, in certain embodiments, antibodies and/or conjugates thereof and/or pharmaceutical compositions thereof are administered to a patient, preferably a human, as a preventative measure against the above various diseases or disorders. Thus, the antibodies and/or conjugates thereof and/or pharmaceutical compositions thereof may be administered as a preventative measure to a patient having a predisposition for a disease characterized by cell migration, cell invasion or cell proliferation, angiogenesis or metastasis. Accordingly, the antibodies and/or conjugates thereof and/or pharmaceutical compositions thereof may be used for the prevention of one disease or disorder and concurrently treating another.

The suitability of the antibodies and/or conjugates thereof and/or pharmaceutical compositions thereof in treating or preventing various diseases or disorders characterized by aberrant vascularization may be assayed by methods described herein and in the art. Accordingly, it is well with the capability of those of skill in the art to assay and use the antibodies and/or conjugates thereof and/or pharmaceutical compositions thereof to treat or prevent diseases or disorders characterized by cell migration, cell invasion or cell proliferation, angiogenesis or metastasis.

### 5.7 Diagnostic Uses and Methods

An antibody and/or a conjugate thereof and/or a pharmaceutical composition thereof is administered to a patient, preferably a human, in a diagnostically effective amount to detect or image a disease such as those listed in Section 5.6 above. Further, antibodies and/or a conjugate thereof and/or pharmaceutical compositions thereof may be used to detect or image diseases or conditions associated with undesired cell migration, invasion or proliferation such as those listed above in Section 5.6 by administering to a subject an diagnostically effective amount of an antibody and/or conjugate thereof and/or a pharmaceutical composition thereof.

Antibodies may be diagnostically labeled and used, for example, to detect peptide-binding ligands or cellular binding sites/receptors (e.g., uPAR) either in the interior or on the surface of a cell. The disposition of the antibody during and after binding may be followed in vitro or in vivo by using an appropriate method to detect the label. Diagnostically labeled antibodies may be utilized in vivo for diagnosis and prognosis, for example, to image occult metastatic foci or for other types of in situ evaluations. For diagnostic applications, antibodies may include bound linker moieties, which are well known to those of skill in the art

In situ detection of the labeled antibody may be accomplished by removing a histological specimen from a subject and examining it by microscopy under appropriate conditions to detect the label. Those of ordinary skill will readily perceive that any of a wide variety of histological methods (such as staining procedures) can be modified in order to achieve such in situ detection.

For diagnostic in vivo radioimaging, the type of detection instrument available is a major factor in selecting a radionuclide. The radionuclide chosen must have a type of decay which is detectable by a particular instrument. In general, any conventional method for visualizing diagnostic imaging can be utilized in accordance with this invention. Another factor in selecting a radionuclide for in vivo diagnosis is that its half-life be long enough so that the label is still detectable at the time of maximum uptake by the target tissue, but short enough so that deleterious irradiation of the host is minimized. In one preferred embodiment, a radionuclide used for in vivo imaging does not emit particles, but produces a large number of photons in a 140-200 keV range, which may be readily detected by conventional gamma cameras.

In vivo imaging may be used to detect occult metastases which are not observable by other methods. The expression of uPAR correlates with progression of diseases in cancer patients such that patients with late stage cancer have higher levels of uPAR in both their primary tumors and metastases. uPAR-targeted imaging could be used to stage tumors non-invasively or to detect other diseases which are associated with the presence of increased levels of uPAR (for example, restenosis that occurs following angioplasty).

Antibodies and/or conjugates thereof may be used in diagnostic, prognostic or research procedures in conjunction with any appropriate cell, tissue, organ or biological sample of the desired animal species. By the term “biological sample” is intended any fluid or other material derived from the body of a normal or diseased subject, such as blood, serum, plasma, lymph, urine, saliva, tears, cerebrospinal fluid, milk, amniotic fluid, bile, ascites fluid, pus and the like. Also included within the meaning of this term is a organ or tissue extract and a culture fluid in which any cells or tissue preparation from the subject has been incubated.

Useful doses are defined as effective amount of antibody and/or conjugate thereof for the particular diagnostic measurement. Thus, an effective amount means an amount sufficient to be detected using the appropriate detection system e.g., magnetic resonance imaging detector, gamma camera, etc. The minimum detectable amount will depend on the ratio of labeled antibody specifically bound to a tumor (signal) to the amount of labeled antibody either bound non-specifically or found free in plasma or in extracellular fluid.

The amount of the diagnostic composition to be administered depends on the precise antibody selected, the disease or condition, the route of administration, and the judgment of the skilled imaging professional. Generally, the amount of antibody needed for detectability in diagnostic use will vary depending on considerations such as age, condition, sex, and extent of disease in the patient, contraindications, if any, and other variables, and is to be adjusted by the individual physician or diagnostician. Dosage can vary from 0.01 mg/kg to 100 mg/kg.

### 5.8 Therapeutic/Prophylactic Administration

The antibodies and/or conjugates thereof and/or pharmaceutical compositions thereof may be advantageously used in human medicine. As previously described in Section 5.6 above, antibodies and/or conjugates thereof and/or pharmaceutical compositions thereof are useful for the treatment or prevention of various diseases or disorders.

When used to treat or prevent the above disease or disorders, antibodies and/or conjugates thereof and/or pharmaceutical compositions thereof may be administered or applied singly, or in combination with other agents. The antibodies and/or conjugates thereof and/or pharmaceutical compositions thereof may also be administered or applied singly, in combination with other pharmaceutically active agents (e.g., other anti-cancer agents, other anti-angiogenic agents such as chelators as zinc, penicillamine, thiomolybdate etc.), including other antibodies described herein.

Methods of treatment and prophylaxis by administration to a patient of a therapeutically effective amount of an antibody and/or conjugates thereof and/or pharmaceutical composition thereof are provided herein. The patient may be an animal, is more preferably, a mammal and most preferably, a human.

The antibodies and/or conjugates thereof and/or pharmaceutical compositions thereof are preferably administered systemically. The antibodies and/or conjugates thereof and/or pharmaceutical compositions thereof may also be administered by any other convenient route, for example, orally, by infusion or bolus injection, by absorption through epithelial or mucocutaneous linings (e.g., oral mucosa, rectal and intestinal mucosa, etc.). Administration can be local. Various delivery systems (e.g., encapsulation in liposomes, microparticles, microcapsules, capsules, etc.) may be used to administer a antibody and/or conjugates thereof and/or pharmaceutical composition thereof. Methods of administration include, but are not limited to, intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. The preferred mode of administration is left to the discretion of the practitioner, and will depend in-part upon the site of the medical condition. In most instances, administration will result in the release of the antibodies and/or conjugates thereof and/or pharmaceutical compositions thereof into the bloodstream.

In specific embodiments, it may be desirable to administer one or more antibodies and/or conjugates thereof and/or pharmaceutical composition thereof locally to the area in need of treatment. This may be achieved, for example, and not by way of limitation, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of cancer or arthritis.

In certain embodiments, it may be desirable to introduce one or more antibodies and/or conjugates thereof and/or pharmaceutical compositions thereof into the central nervous system by any suitable route, including intraventricular, intrathecal and epidural injection. Intraventricular injection may be facilitated by an intraventricular catheter, for example, attached to a reservoir, such as an Ommaya reservoir.

An antibody and/or conjugate thereof and/or pharmaceutical composition hereof may also be administered directly to the lung by inhalation. For administration by inhalation, an antibody and/or conjugate thereof and/or pharmaceutical composition thereof may be conveniently delivered to the lung by a number of different devices. For example, a Metered Dose Inhaler (“MDI”), which utilizes canisters that contain a suitable low boiling propellant, (e.g. dichlorodifluoromethane, trichlorofluoromethane, dichlorotetrafluoroethane, carbon dioxide or any other suitable gas) may be used to deliver antibodies and/or conjugates thereof directly to the lung.

Alternatively, a Dry Powder Inhaler (“DPI”) device may be used to administer an antibody and/or conjugate thereof and/or pharmaceutical composition thereof to the lung. DPI devices typically use a mechanism such as a burst of gas to create a cloud of dry powder inside a container, which may then be inhaled by the patient. DPI devices are also well known in the art. A popular variation is the multiple dose DPI (“MDDPI”) system, which allows for the delivery of more than one therapeutic dose. For example, capsules and cartridges of gelatin for use in an inhaler or insufflator may be formulated containing a powder mix of an antibody and/or conjugate thereof and a suitable powder base such as lactose or starch for these systems.

Another type of device that may be used to deliver an antibody and/or conjugate thereof and/or pharmaceutical composition hereof to the lung is a liquid spray device supplied, for example, by Aradigm Corporation (Hayward, Calif.). Liquid spray systems use extremely small nozzle holes to aerosolize liquid drug formulations that may then be directly inhaled into the lung.

In one embodiment, a nebulizer is used to deliver a antibody and/or conjugate thereof and/or pharmaceutical composition thereof to the lung. Nebulizers create aerosols from liquid drug formulations by using, for example, ultrasonic energy to form fine particles that may be readily inhaled (see e.g., Verschoyle et al., British J. Cancer, 1999, 80, Suppl. 2, 96, which is herein incorporated by reference). Examples of nebulizers include devices supplied by Batelle Pulmonary Therapeutics (Columbus, Ohio) (See, Armer et al., U.S. Pat. No. 5,954,047; van der Linden et al., U.S. Pat. No. 5,950,619; van der Linden et al., U.S. Pat. No. 5,970,974).

In another embodiment, an electrohydrodynamic (“EHD”) aerosol device is used to deliver an antibody and/or conjugate thereof and/or pharmaceutical composition thereof to the lung. EHD aerosol devices use electrical energy to aerosolize liquid drug solutions or suspensions (see e.g., Noakes et al., U.S. Pat. No. 4,765,539). EHD aerosol devices may more efficiently deliver drugs to the lung than existing pulmonary delivery technologies.

In another embodiment, the antibodies and/or conjugates thereof and/or pharmaceutical compositions thereof can be delivered in a vesicle, in particular a liposome (see Langer, 1990, Science, 249:1527-1533; Treat et al., in “Liposomes in the Therapy of Infectious Disease and Cancer,” Lopez-Berestein and Fidler (eds.), Liss, N.Y., pp. 353-365 (1989); see generally “Liposomes in the Therapy of Infectious Disease and Cancer,” Lopez-Berestein and Fidler (eds.), Liss, N.Y., pp. 353-365 (1989)).

### 5.9 Pharmaceutical Compositions

The present pharmaceutical compositions contain a therapeutically or diagnostically effective amount of one or more antibodies and/or conjugates thereof, preferably, in purified form, together with a suitable amount of a pharmaceutically acceptable vehicle, so as to provide the form for proper administration to a patient. When administered to a patient, the antibodies and/or conjugates thereof and pharmaceutically acceptable vehicles are preferably sterile. Water is a preferred vehicle when the antibodies and/or conjugates thereof are administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present pharmaceutical compositions, if desired, can also contain minor amounts of wetting or emulsifying agents, or pH buffering agents. In addition, auxiliary, stabilizing, thickening, lubricating and coloring agents may be used.

Pharmaceutical compositions comprising a antibody and/or conjugate thereof may be manufactured by means of conventional mixing, dissolving, granulating, dragee-making, levigating, emulsifying, encapsulating, entrapping or lyophilizing processes. Pharmaceutical compositions may be formulated in conventional manner using one or more physiologically acceptable carriers, diluents, excipients or auxiliaries, which facilitate processing of antibodies and/or conjugates thereof into preparations which can be used pharmaceutically. Proper formulation is dependent upon the route of administration chosen.

The present pharmaceutical compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable vehicle is a capsule (see e.g., Grosswald et al., U.S. Pat. No. 5,698,155). Other examples of suitable pharmaceutical vehicles have been described in the art (see Remington's Pharmaceutical Sciences, Philadelphia College of Pharmacy and Science, 19th Edition, 1995).

For topical administration, antibodies and/or conjugates thereof may be formulated as solutions, gels, ointments, creams, suspensions, etc. as is well-known in the art. Systemic formulations include those designed for administration by injection, e.g., subcutaneous, intravenous, intramuscular, intrathecal or intraperitoneal injection, as well as those designed for transdermal, transmucosal, oral or pulmonary administration. Systemic formulations may be made in combination with a further active agent that improves mucociliary clearance of airway mucus or reduces mucous viscosity. These active agents include, but are not limited to, sodium channel blockers, antibiotics, N-acetyl cysteine, homocysteine and phospholipids.

In some embodiments, antibodies and/or conjugates thereof are formulated in accordance with routine procedures as a pharmaceutical composition adapted for intravenous administration to human beings. Typically, antibodies and/or conjugates thereof for intravenous administration are solutions in sterile isotonic aqueous buffer. For injection, antibodies and/or conjugates thereof may be formulated in aqueous solutions, preferably, in physiologically compatible buffers such as Hanks' solution, Ringer's solution, or physiological saline buffer. The solution may contain formulatory agents such as suspending, stabilizing and/or dispersing agents. When necessary, the pharmaceutical compositions may also include a solubilizing agent. Pharmaceutical compositions for intravenous administration may optionally include a local anesthetic such as lignocaine to ease pain at the site of the injection. Generally, the ingredients are supplied either separately or mixed together in unit dosage form, for example, as a lyophilized powder or water free concentrate in a hermetically sealed container such as an ampoule or sachette indicating the quantity of active agent. When antibodies and/or conjugates thereof are administered by infusion, they can be dispensed, for example, with an infusion bottle containing sterile pharmaceutical grade water or saline. When antibodies and/or conjugates thereof are administered by injection, an ampoule of sterile water for injection or saline can be provided so that the ingredients may be mixed prior to administration.

For transmucosal administration, penetrants appropriate to the barrier to be permeated are used in the formulation. Such penetrants are generally known in the art.

Pharmaceutical compositions for oral delivery may be in the form of tablets, lozenges, aqueous or oily suspensions, granules, powders, emulsions, capsules, syrups, or elixirs, for example. Orally administered pharmaceutical compositions may contain one or more optionally agents, for example, sweetening agents such as fructose, aspartame or saccharin; flavoring agents such as peppermint, oil of wintergreen, or cherry coloring agents and preserving agents, to provide a pharmaceutically palatable preparation. Oral compositions can include standard vehicles such as mannitol, lactose, starch, magnesium stearate, sodium saccharine, cellulose, magnesium carbonate, etc. Such vehicles are preferably of pharmaceutical grade.

For oral liquid preparations such as, for example, suspensions, elixirs and solutions, suitable carriers, excipients or diluents include water, saline, alkyleneglycols (e.g., propylene glycol), polyalkylene glycols (e.g., polyethylene glycol) oils, alcohols, slightly acidic buffers between pH 4 and pH 6 (e.g., acetate, citrate, ascorbate at between about 5.0 mM to about 50.0 mM), etc. Additionally, flavoring agents, preservatives, coloring agents, bile salts, acylcarnitines and the like may be added.

For buccal administration, the pharmaceutical compositions may take the form of tablets, lozenges, etc. formulated in conventional manner.

Liquid drug formulations suitable for use with nebulizers and liquid spray devices and EHD aerosol devices will typically include an antibody and/or conjugates thereof with a pharmaceutically acceptable vehicle. Preferably, the pharmaceutically acceptable vehicle is a liquid such as alcohol, water, polyethylene glycol or a perfluorocarbon. Optionally, another material may be added to alter the aerosol properties of the solution or suspension of antibodies and/or conjugates thereof. Preferably, this material is liquid such as an alcohol, glycol, polyglycol or a fatty acid. Other methods of formulating liquid drug solutions or suspension suitable for use in aerosol devices are known to those of skill in the art (see, e.g., Biesalski, U.S. Pat. No. 5,112,598; Biesalski, U.S. Pat. No. 5,556,611).

A antibody and/or conjugates thereof may also be formulated in rectal or vaginal pharmaceutical compositions such as suppositories or retention enemas, e.g., containing conventional suppository bases such as cocoa butter or other glycerides.

### 5.10 Doses

A antibody and/or conjugates thereof, or pharmaceutical compositions thereof, will generally be used in an amount effective to achieve the intended purpose. For use to treat or prevent diseases or disorders characterized by aberrant vascularization the antibodies and/or conjugates thereof and/or pharmaceutical compositions thereof, are administered or applied in a therapeutically effective amount. For use to detect diseases or disorders characterized by aberrant vascularization the antibodies and/or conjugates thereof and/or pharmaceutical compositions thereof, are administered or applied in a diagnostically effective amount.

The amount of a antibody and/or conjugates thereof that will be effective in the treatment, prevention or detection of a particular disorder or condition disclosed herein will depend on the nature of the disorder or condition, and can be determined by standard clinical techniques known in the art as previously described. In addition, in vitro or in vivo assays may optionally be employed to help identify optimal dosage ranges. The amount of a antibody and/or conjugates thereof administered will, of course, be dependent on, among other factors, the subject being treated, the weight of the subject, the severity of the affliction, the manner of administration and the judgment of the prescribing physician.

For example, the dosage may be delivered in a pharmaceutical composition by a single administration, by multiple applications or controlled release. In one embodiment, the antibodies and/or conjugates thereof are delivered by oral sustained release administration. Preferably, in this embodiment, the antibodies and/or conjugates thereof are administered twice per day (more preferably, once per day). Dosing may be repeated intermittently, may be provided alone or in combination with other drugs and may continue as long as required for effective treatment of the disease state or disorder.

Suitable dosage ranges for oral administration are dependent on the potency of the drug, but are generally about 0.001 mg to about 200 mg of a antibody and/or conjugates thereof per kilogram body weight. Dosage ranges may be readily determined by methods known to the artisan of ordinary skill.

Suitable dosage ranges for intravenous (i.v.) administration are about 0.01 mg to about 100 mg per kilogram body weight. Suitable dosage ranges for intranasal administration are generally about 0.01 mg/kg body weight to about 1 mg/kg body weight. Suppositories generally contain about 0.01 milligram to about 50 milligrams of a antibody and/or conjugates thereof per kilogram body weight and comprise active ingredient in the range of about 0.5% to about 10% by weight. Recommended dosages for intradermal, intramuscular, intraperitoneal, subcutaneous, epidural, sublingual or intracerebral administration are in the range of about 0.001 mg to about 200 mg per kilogram of body weight. Effective doses may be extrapolated from dose-response curves derived from in vitro or animal model test systems. Such animal models and systems are well-known in the art.

The antibodies and/or conjugates thereof are preferably assayed in vitro and in vivo, as described above, for the desired therapeutic, prophylactic or diagnostic activity, prior to use in humans. For example, in vitro assays can be used to determine whether administration of a specific antibody and/or conjugates thereof or a combination of antibodies and/or conjugates thereof is preferred for treating, preventing or diagnosing cancer. The antibodies and/or conjugates thereof may also be demonstrated to be effective and safe using animal model systems.

Preferably, a therapeutically effective dose of a antibody and/or conjugates thereof described herein will provide therapeutic benefit without causing substantial toxicity. Similarly, a diagnostically effective dose of an antibody and/or conjugates thereof described herein will provide diagnostic benefit without causing substantial toxicity. Toxicity of antibodies and/or conjugates thereof may be determined using standard pharmaceutical procedures and may be readily ascertained by the skilled artisan. The dose ratio between toxic and therapeutic effect is the therapeutic index. A antibody and/or conjugates thereof will preferably exhibit particularly high therapeutic indices in treating disease and disorders. The dosage of a antibody and/or conjugates thereof described herein will preferably be within a range of circulating concentrations that include an effective therapeutic or diagnostic does dose with little or no toxicity.

### 5.11 Combination Therapy

In certain embodiments, the antibodies and/or conjugates thereof and/or pharmaceutical compositions thereof can be used in combination therapy with at least one other therapeutic agent. The antibodies and/or conjugates thereof and/or pharmaceutical composition thereof and the therapeutic agent can act additively or, more preferably, synergistically. In some embodiments, antibodies and/or conjugates thereof and/or pharmaceutical compositions thereof are administered concurrently with the administration of another therapeutic agent, which may be part of the same pharmaceutical composition or a different pharmaceutical composition. In another embodiment, a pharmaceutical composition of antibodies and/or conjugates thereof is administered prior or subsequent to administration of another therapeutic agent.

In particular, in other embodiments, the antibodies and/or conjugates thereof and/or pharmaceutical compositions thereof can be used in combination therapy with other chemotherapeutic agents (e.g., alkylating agents (e.g., nitrogen mustards (e.g., cyclophosphamide, ifosfamide, mechlorethamine, melphalen, chlorambucil, hexamethylmelamine, thiotepa), alkyl sulfonates (e.g., busulfan), nitrosoureas, triazines), antimetabolites (e.g., folic acid analogs, pyrimidine analogs (e.g., fluorouracil, floxuridine, cytosine arabinoside, etc.), purine analogs (e.g., mercaptopurine, thiogunaine, pentostatin, etc.), natural products (e.g., vinblastine, vincristine, etoposide, tertiposide, dactinomycin, daunorubicin, doxurubicin, bleomycin, mithrmycin, mitomycin C, L-asparaginase, interferon alpha), platinum coordination complexes (e.g., cis-platinum, carboplatin, etc.), mitoxantrone, hydroxyurea, procarbazine, hormones and antagonists (e.g., prednisone, hydroxyprogesterone caproate, medroxyprogesterone acetate, megestrol acetate, diethylstilbestrol, ethinyl estradiol, tamoxifen, testosterone propionate, fluoxymesterone, flutamide, leuprolide, etc.), anti-angiogenesis agents or inhibitors (e.g., angiostatin, retinoic acids and paclitaxel, estradiol derivatives, thiazolopyrimidine derivatives, etc.), apoptosis-inducing agents (e.g., antisense nucleotides that block oncogenes which inhibit apoptosis, tumor suppressors, TRAIL, TRAIL polypeptide, Fas-associated factor 1, interleukin-1β-converting enzyme, phosphotyrosine inhibitors, RXR retinoid receptor agonists, carbostyril derivatives, etc.) and chelators (penicillamine, zinc, trientine, etc.).

### 5.12 Therapeutic Kits

Therapeutic kits comprising the antibodies and/or conjugates thereof or pharmaceutical compositions thereof are also provided. The therapeutic kits may also contain other compounds (e.g., chemotherapeutic agents, natural products, hormones or antagonists, anti-angiogenesis agents or inhibitors, apoptosis-inducing agents or chelators) or pharmaceutical compositions of these other compounds.

Therapeutic kits may have a single containers which contains the antibody and/or conjugates thereof or pharmaceutical compositions thereof with or without other components (e.g., other compounds or pharmaceutical compositions of these other compounds) or may have distinct container for each component. Therapeutic kits include antibodies and/or conjugates thereof and/or a pharmaceutical composition thereof packaged for use in combination with the co-administration of a second compound (preferably, a chemotherapeutic agent, a natural product, a hormone or antagonist, a anti-angiogenesis agent or inhibitor, a apoptosis-inducing agent or a chelator) and/or a pharmaceutical composition thereof. The components of the kit may be pre-complexed or each component may be in a separate distinct container prior to administration to a patient.

The components of the kit may be provided in one or more liquid solutions, preferably, an aqueous solution, more preferably, a sterile aqueous solution. The components of the kit may also be provided as solids, which may be converted into liquids by addition of suitable solvents, which are preferably provided in another distinct container.

The container of a therapeutic kit may be a vial, test tube, flask, bottle, syringe, or any other means of enclosing a solid or liquid. Usually, when there is more than one component, the kit will contain a second vial or other container, which allows for separate dosing. The kit may also contain another container for a pharmaceutically acceptable liquid.

Preferably, a therapeutic kit will contain apparatus (e.g., one or more needles, syringes, eye droppers, pipette, etc.), which enables administration of the components of the kit.

The following examples describe in detail, preparation of antibodies and/or conjugates thereof and methods for assaying for biological activity. It will be apparent to those skilled in the art that many modifications, both to materials and methods, may be practiced without departing from the scope of the invention.

### 6.1 EXAMPLE 1

### Expression and Purification of the Amino Terminal Fragment of Urokinase

The amino terminal fragment of urokinase (amino acids 1-143) was cloned and expressed in Drosophila Schneider S2 cells. Cells were induced to express recombinant protein with copper (0.5 mM) for 7 days. Culture supernatants were collected and clarified by centrifugation and filtration. After addition of protease inhibitors, the amino terminal fragment of urokinase was purified by ion exchange chromatography on DEAE-Sepharose, pH 7.5 and was further purified using reverse phase-HPLC.

### 6.2 EXAMPLE 2

### Immunization and Preparation of Monoclonal Antibodies

Balb/c mice were injected with the amino terminal fragment of urokinase prepared in Example 6.1 and immune response monitored by ELISA. Based on the ELISA data, hybridomas were generated by fusing spleen cells with the myeloma cell line P3x63Ag8.653. Frozen stocks of 10 parental hybridomas were made and 5 of the hybridomas subjected to limiting dilution. Tissue culture supernatants from these monoclonal antibodies were then assayed for activity in an ELISA assay and the isotype of each antibody determined using IsoStrips (Roche). Sufficient antibody for animal and other studies was produced from ascites. Processed ascites was further purified on protein A Sepharose and the purity of the final material (>95%) determined by HPLC. Finally, the identity of the purified antibody was further characterized by isoelectric focusing and isotype determination. An extensive panel of monoclonal antibodies (all IgG₁, κ) specific for the amino terminal domain of urokinase was generated (data not shown).

### 6.3 EXAMPLE 3

### Characterization of Monoclonal Antibodies

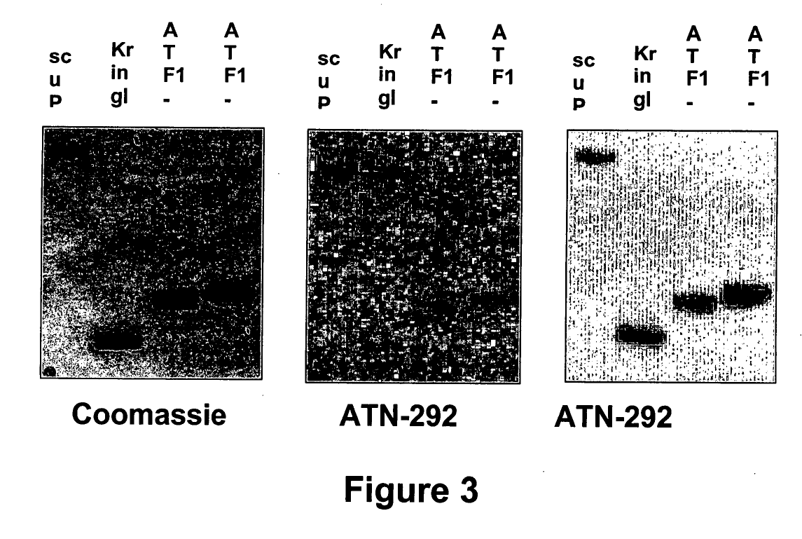

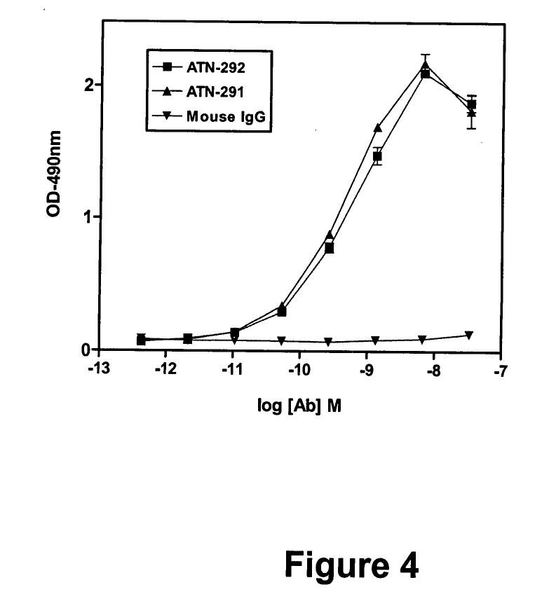

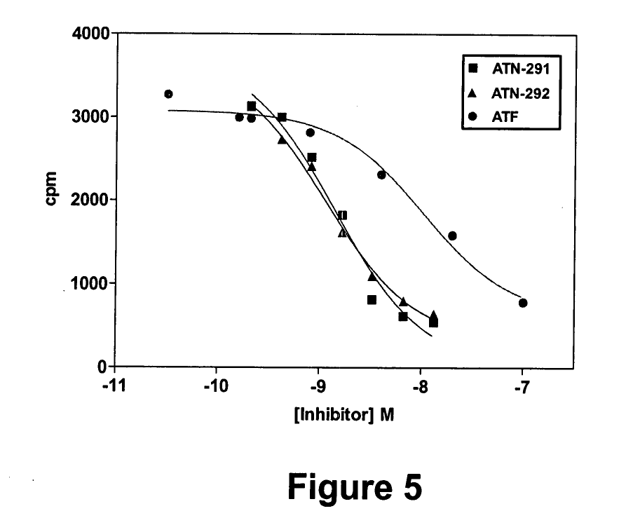

Two of the antibodies, provided by Example 6.2, ATN-291 and ATN-292, were extensively characterized and produced in sufficient quantities for in vivo experiments. Initial epitope mapping experiments were performed using western blots. Recombinant proteins (i.e., scu, Kringle, amino terminal fragment 1-135 and amino terminal fragment 1-143) were resolved by SDS-PAGE and transferred to PVDF membranes. As shown in FIG. 3, ATN-292 specifically bound recombinant, amino terminal fragment 1-135, amino terminal fragment 1-143, but not urokinase Kringle domain which indicates that ATN-292 recognized the growth factor domain of urokinase. In contrast, ATN-291 specifically recognized the uPA Kringle domains as can be seen in FIG. 3. Direct binding experiments were used to determine the K_{D} of the antibodies. As shown in FIG. 4, both ATN-291 and ATN-292 bound urokinase with high affinity with K_{D}'s of ˜0.3 and ˜0.5 nM respectively. ATN-291 and ATN-292 were tested for their ability to inhibit binding of ATF to HeLa cells. As shown in FIG. 5, both antibodies inhibited binding of ATF with an IC₅₀ of ˜2 nM. This is expected for ATN-292 which is specific for the GFD (uPAR binding) domain of uPA. ATN-291 is specific for the Kringle domain and inhibition is therefore probably due to steric hindrance.

### 6.4 EXAMPLE 4

### Inhibition of Tumor Growth by Monoclonal Antibodies

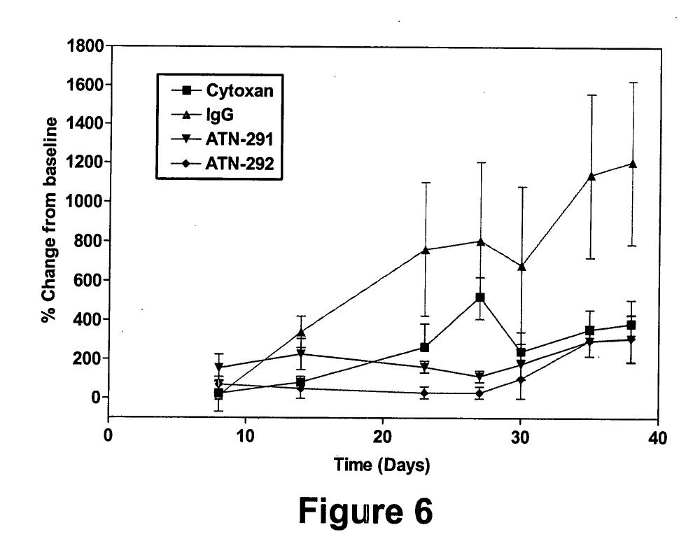

Antibodies were tested for their ability to inhibit tumor growth in an MDA MB 231 breast carcinoma model. Balb/c nu/nu mice were injected with 7×10⁵ MDA MB 231 breast carcinoma cells and tumors staged to 35 mm³. Animals were divided randomly into treatment groups of 10 and treated with antibodies, 10 mg/kg (200 □g/mouse), three times per week, IP. As shown in FIG. 6, both ATN-291 and ATN-292 significantly inhibited tumor growth in this model when compared to an isotype matched control antibody.

### 6.5 EXAMPLE 5

### Internalization of Monoclonal Antibodies

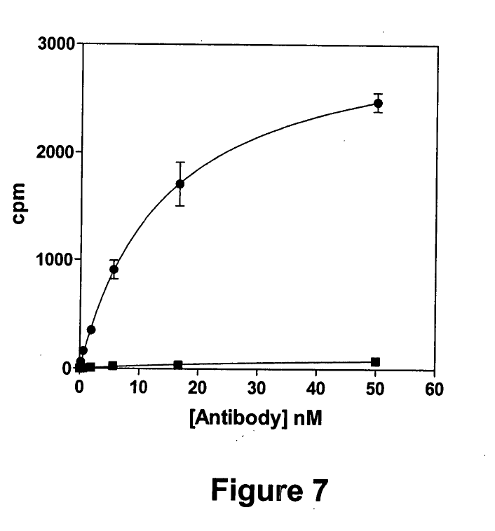

The potential of antibodies directed towards the amino terminal fragment of urokinase to deliver cytotoxic agents was determined by internalization experiments performed with [¹²⁵I]-labeled antibodies and MDA-MB-231 cells. MDA-MB-231 cells express both uPA and uPAR. Acid-washing experiments revealed that a significant proportion of all uPAR receptors on the surface of these cells are occupied by uPA (data not shown). Confluent monolayers of MDA-MB-231 cells in 24-well plates were incubated with increasing concentrations of [¹²⁵I]-ATN-291 (□oe) or [125I]-ATN-292 (□ᵢ) at room temperature for one hour FIG. 7). Cells were washed extensively with PBS/Tween-20 and bound material was solubilized with 1 M NaOH. Non-specific binding was determined in the presence of a 20-fold excess of unlabeled antibody. The above experiments revealed that ATN-291, but not ATN-292, can bind to receptor-bound uPA on the surface of MDA-MB-231 cells with high affinity (FIG. 7). These results are consistent with the epitope mapping studies of Example 6.2 that demonstrated that ATN-292 bound the growth factor domain of uPA. This epitope is essential for uPA binding to uPAR and is therefore masked when the ligand is bound to the receptor. In contrast, ATN-291 recognizes the Kringle domain of uPA, which is involved in stabilizing the uPA-uPAR interaction but is not essential for uPA binding, and is partially exposed even when the ligand is bound to its receptor.

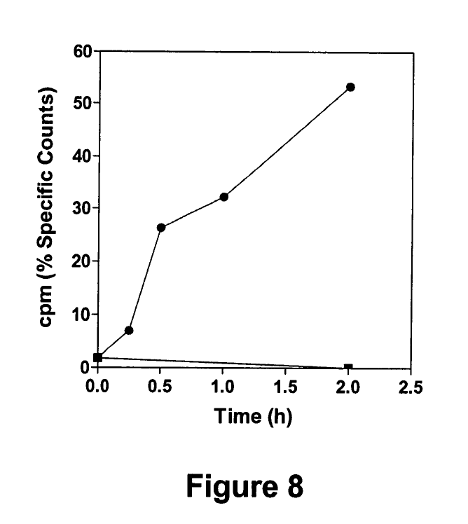

Internalization of [¹²⁵I]-ATN-291 was determined using standard techniques. Briefly, MDA-MB-231 cells were incubated with labeled ATN-291 for 2 h at 4° C. Cells were washed extensively and the final wash replaced with binding buffer pre-warmed to 37° C. Cells were incubated at 37° C. and at various times the cellular distribution of [¹²⁵I]-ATN-291 was determined as follows: supernatant was collected and fractionated by TCA precipitation, membrane bound antibody was removed by acid-wash and antibody associated with the cell lysate was recovered by lysis of the adherent, acid-washed cells. Degradation of ATN-291 is represented as a % of the total specific counts bound by the cells. A significant, time-dependant increase in non-precipitable (degraded) ATN-291 was observed when cells were incubated at 37° C. (FIG. 8). In contrast, no degradation was observed when cells with bound [¹²⁵I]-ATN-291 were maintained at 4° C.

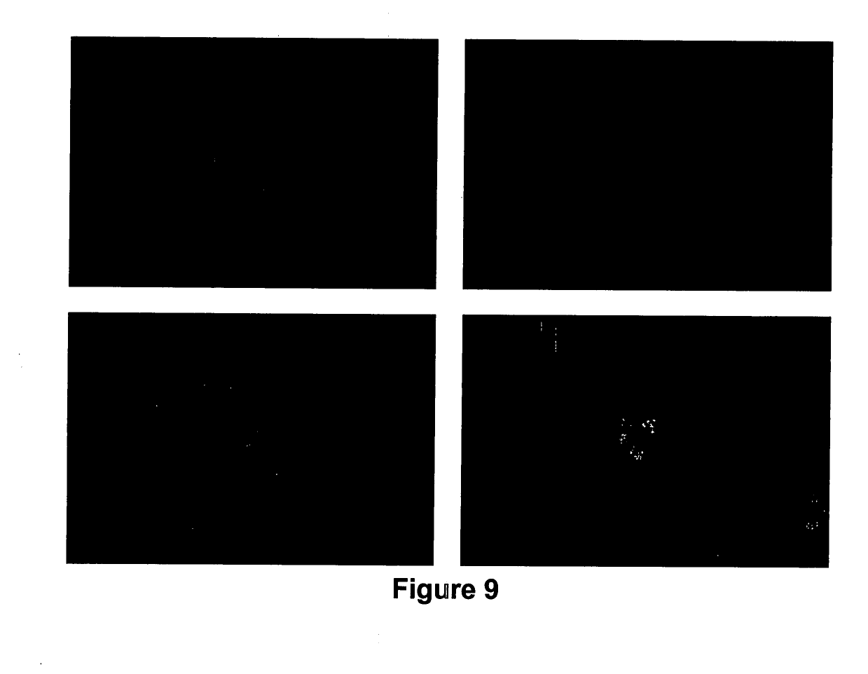

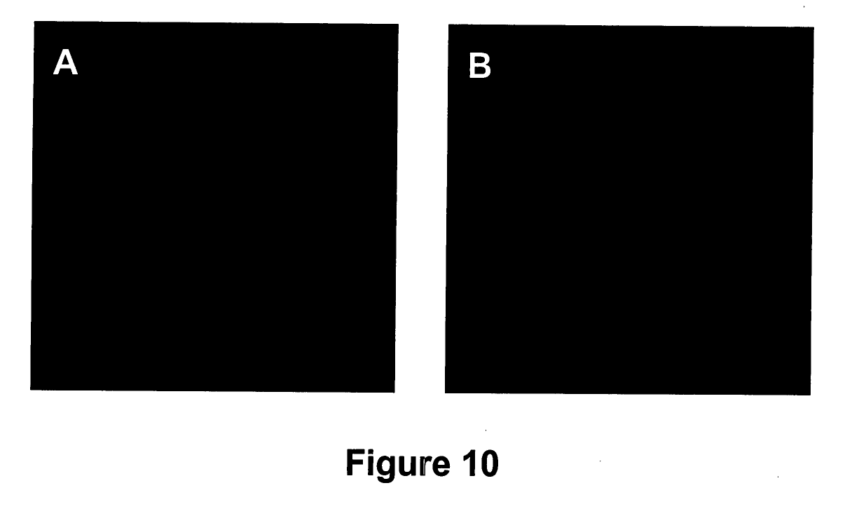

Internalization of ATN-291 by MDA-MB-231 cells was confirmed by characterizing the cellular distribution of the antibody using either a FITC-conjugated secondary antibody (FIG. 9) or ATN-291-CypHer-5 conjugates (FIG. 10). Cells were incubated with 10 μg/ml ATN-291 at either 4° C. (panel C) or 37° C. (panel D) for 2 h. After incubation, cells were washed, fixed and permeabilized and ATN-291 detected with a goat anti-mouse-FITC conjugated antibody. Control cells were incubated with mIgG (panel A) or secondary antibody alone (panel B). Cell nuclei were counter-stained with DAPI. Cells incubated with ATN-291 at 4° C. demonstrated a very diffuse pattern of fluorescence (FIG. 9C). In contrast, cells incubated with ATN-291 at 37° C. showed evidence of perinuclear staining, consistent with internalization of the antibody into a golgi-like compartment (FIG. 9D).

CypHer 5 is a novel, red-excitable, pH-sensitive cyanine dye derivative. CypHer 5 is non-fluorescent at pH 7.4 and is maximally fluorescent at pH 5.5, thus providing a useful tool to determine the internalization of labeled molecules into internal acidic endosomes. ATN-291 was labeled with CypHer 5 (Amersham Biosciences) according to the manufacturer's instructions. Absorbance at OD-480 nm indicated that, on average, ˜1.6 Cypher 5 molecules were conjugated to each antibody molecule. Cells were incubated with 1 μM ATN-291-CY5 at 37° C. for 2 h (panel A) or 4 h (panel B). Cells incubated with CypHer 5 labeled ATN-291 showed a time-dependent increase in red-fluorescence (FIGS. 10A and 10B). The above data strongly suggests that ATN-291 binds to receptor bound uPA with high affinity and is then internalized and degraded by MDA-MB-231 cells.

### 6.6 EXAMPLE 6

### Synthesis of Doxorubicin Conjugate

Treatment of doxorubicin hydrochloride (1) with glutaric anhydride in the presence of Hunig's base gave the acid 2, which was converted in situ to the corresponding N-hydroxysuccinyl ester 3 with N-hydroxysuccinimide and EEDQ in DMF at 0° C. for 1 hour (See FIG. 9). This solution was added to ATN-291 in PBS (pH 8.1, 2 mL of 3 mg/mL) and the resulting red solution was stored at 4° C. for 19 hours. The volume of the reaction was adjusted to 3 mL with PBS, pH 8.1, and the conjugated antibody purified from free doxorubicin by size exclusion chromatography using a PD-10column. The number of doxorubicin molecules conjugated to ATN-291 was determined by MALDI-TOF.

### 6.7 EXAMPLE 7

### Characterization of Doxorubicin Conjugate

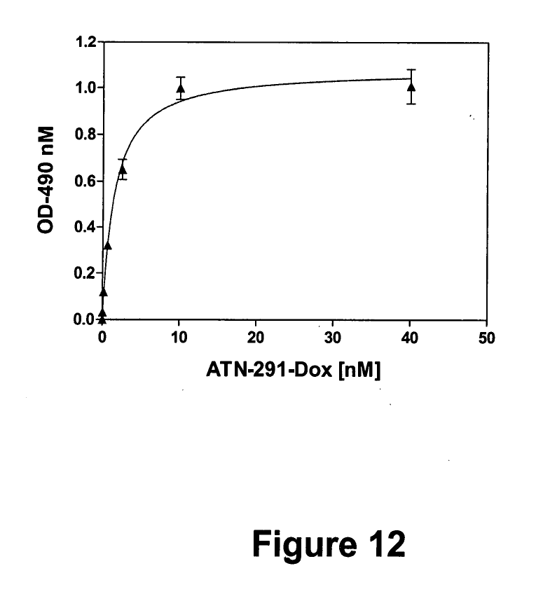

To confirm that the antibody component of the ATN-291-Dox conjugates was still functional, and still bound uPA with high affinity, ELISA assays were performed. As shown in FIG. 12, an ATN-291-Dox conjugate containing an average of 4 Dox molecules per antibody, bound uPA with an affinity similar to that of the non-conjugated antibody.

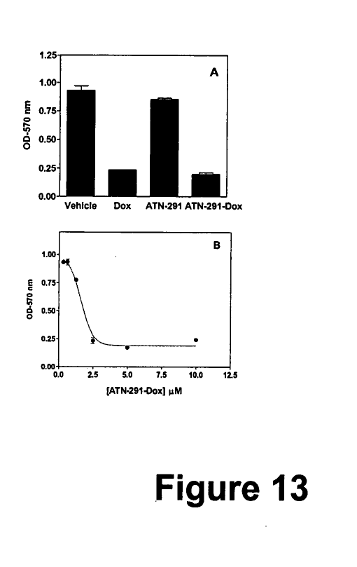

The ability of ATN-291-Dox to inhibit proliferation of MDA-MB-231 cells was tested in an MTT assay. As shown in FIG. 13A, ATN-291 (10 μM) had no significant effect on cell proliferation whereas 10 μM ATN-291-Dox, or 10 μM Dox alone, significantly inhibited proliferation of MDA-MB-231 cells. A dose titration was performed with the ATN-291-Dox conjugate. As shown in FIG. 10B the conjugate inhibited proliferation with an IC-50 of ˜1.6 μM.

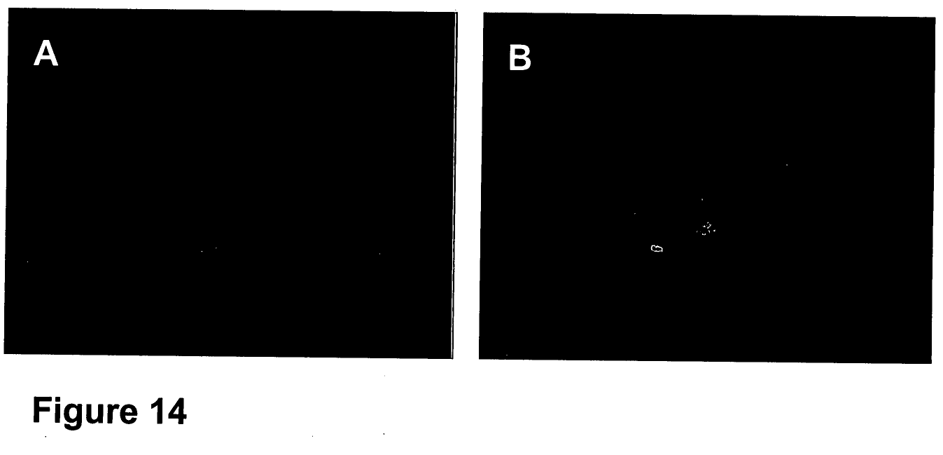

To further characterize the ATN-291-Dox conjugates the distribution of Dox was monitored by fluorescence microscopy. MDA-MB-231 cells grown on glass chamber slides were incubated with either 1.6 μM Dox or 1.6 μM ATN-291-Dox for 24 h under the same conditions used for the MTT assay. Following incubation, the supernatant was removed and the cells washed extensively with PBS. Cells were fixed in paraformaldehyde, mounted and observed by fluorescence microscopy. As shown in FIG. 14A Dox was primarily localized to the nucleus of treated cells. In contrast, ATN-292-Dox treated cells showed a distinct, perinuclear pattern of staining (FIG. 14B). These data suggest that ATN-291-Dox is internalized by cells and is translocated via a golgi-like compartment, presumably before being degraded in lysosomes. Similar results have been previously reported for other internalized antibodies including the anti-CD22 antibody, LL2.

### 6.8 EXAMPLE 8

### Synthesis of Doxorubicin Diimide Conjugate

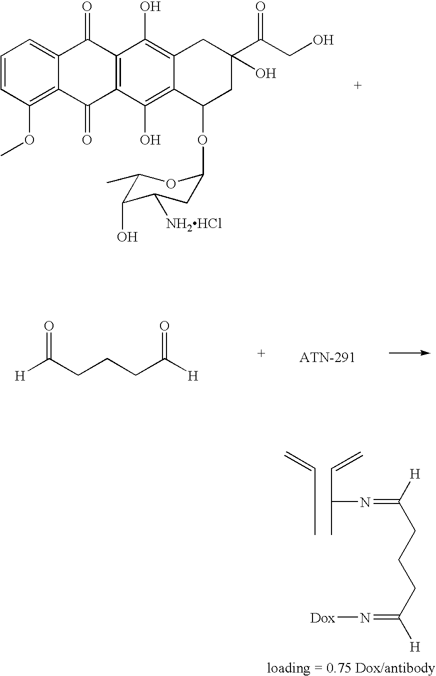

Doxorubicin hydrochloride (0.5 mg, 0.00086 mmol) in 28 μL of deionized water was added to ATN-291 in PBS pH 8.1 (2 mL, 3 mg/mL, 0.00004 mmol) at room temperature. Glutaric dialdehyde (0.1% in water, 200 μL, 0.0002 mmol) was added slowly and the reaction mixture was stirred for 15 minutes in the dark. The conjugated antibody was separated from unreacted doxorubicin and glutaric dialdehyde with a PD-10 column, eluting with 4 mL of PBS pH 8.1. The conjugated antibody then underwent a buffer exchange with water and the loading was determined by MALDI-TOF. The product was obtained as a pink solution: MALDI-TOF m/z (M avg) 151178.

### 6.9 EXAMPLE 9

### Synthesis of Camptothecin Conjugate

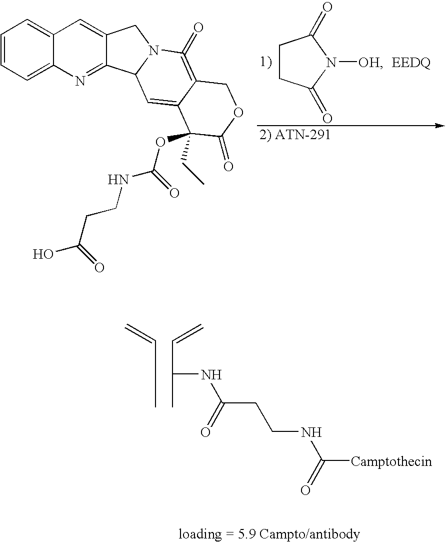

Camptothecin-linker compound (12 mg, 0.026 mmol) and N-hydroxysuccinimide (4.6 mg, 0.040 mmol) were dissolved in DMF (2 mL) and cooled in an ice bath. EEDQ (7.7 mg, 0.031 mmol) was added and the yellow solution was stirred for one hour. 306 μL (0.0040 mmol, 100 eq) of this solution was added to ATN-291 (2 mL, 3 mg/mL in PBS pH 8.1, 0.00004 mmol) and the reaction mixture was stored in the refrigerator for 21 hours. The conjugated antibody was separated from unreacted 1 with a PD-10 column, eluting with 4 mL of PBS pH 8.1. The conjugated antibody then underwent a buffer exchange with water and the loading was determined by MALDI-TOF. The product was obtained as a light yellow solution: MALDI-TOF m/z (M avg) 152845.

### 6.10 EXAMPLE 10

### Synthesis of Doxurubicin Thioether Hydrazone Conjugate

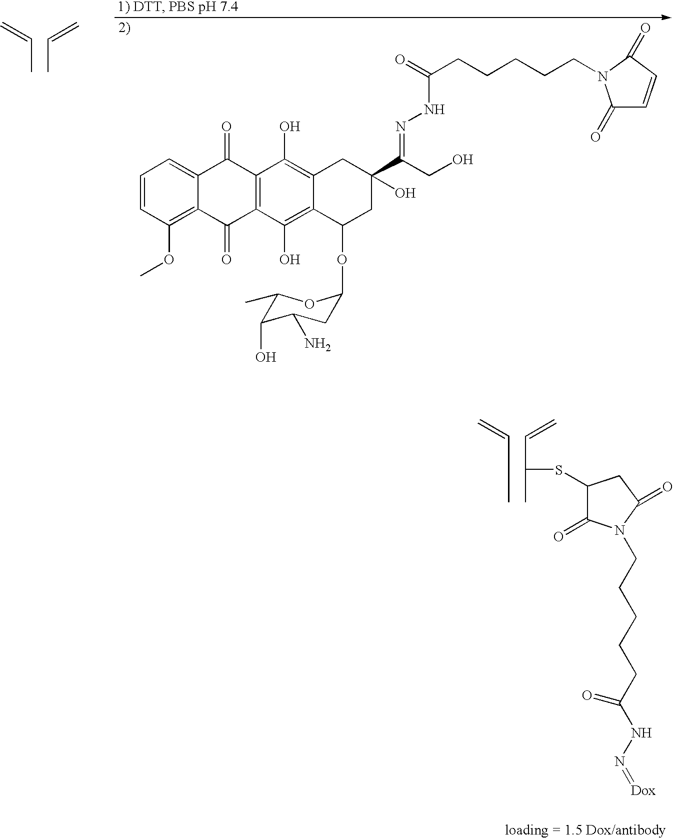

ATN-291 (2 mL of a 5 mg/mL solution in PBS pH 7.4, 0.000067 mmol) was degassed with nitrogen and then a degassed 34.4 mM solution of dithiothreitol in PBS pH 7.4 (14 μL, 0.00048 mmol) was added. The reaction mixture was stirred at 37° C. for three hours. The reduced antibody was purified with a PD-10 column, eluting with 4 mL of PBS pH 7.4. The thiol concentration was determined to be 75 μM with Eliman's reagent (4.5 mL of solution). The doxorubicin-hydrazone compound (0.33 mg, 0.00044 mmol) as a solution in water was added to the reduced antibody at 0° C. and the reaction mixture was stirred for 30 minutes. The conjugated antibody was purified with a PD-10 column, eluting with 4 mL of PBS pH 7.4. The conjugated antibody then underwent a buffer exchange with water and the loading was determined by MALDI-TOF. The product was obtained as a pink solution: MALDI-TOF m/z (M avg) 151119.

Finally, it should be noted that there are alternative ways of implementing the present invention. Accordingly, the present embodiments are to be considered as illustrative and not restrictive, and the invention is not to be limited to the details given herein, but may be modified within the scope and equivalents of the appended claims. All publications and patents cited herein are incorporated by reference in their entirety.

1. An antibody which binds to the amino terminal fragment of urokinase.

2. The antibody of claim 1, wherein the amino terminal fragment is amino acids 1-143 of SEQ ID NO 1.

3. The antibody of claim 1, which binds to the growth factor domain of urokinase.

4. The antibody of claim 3 wherein the growth factor domain is amino acids 1-48 of SEQ ID NO 1.

5. The antibody of claim 1 which binds to the Kringle domain of urokinase.

6. The antibody of claim 5, wherein the Kringle domain is amino acids 49-135 of SEQ ID NO 1.

7. The antibody of claim 2 which binds to amino acids 136-143 of SEQ ID NO 1.

8. The antibody of claim 1, wherein the antibody is a monoclonal antibody.

9. The antibody of claim 1, wherein the antibody is fused to a protein toxin.

10. The antibody of claim 9, wherein the toxin is Pseudomonas exotoxin.

11. The antibody of claim 1, wherein the antibody is an IgG1 antibody.

12. The antibody of claim 11, wherein the antibody is a κ antibody.

13. The antibody of claim 1, wherein the antibody is conjugated to a therapeutic agent.

14. The antibody of claim 13, wherein the therapeutic agent is a cytotoxic cancer agent.

15. The antibody of claim 14, wherein the cytotoxic cancer agent is a taxane, a camptothecin, an epithilone or taxol.

16. The antibody of claim 15, wherein the cytotoxic cancer agent is doxorubicin.

17. The antibody of claim 13, wherein the therapeutic agent is a radionuclide.

18. The antibody of claim 1, wherein the antibody is conjugated to a diagnostic agent.

19. The antibody of claim 18, wherein the diagnostic agent is a radionuclide, an agent imageable by positron emission tomography, an magnetic resonance imaging agent, a fluorescent agent, a fluorogen, a chromophore, a chromogen, a phosphorescent agent, a chemiluminescent agent or a bioluminescent agent.

20. The antibody of any one of claims 1, 13 or 18 wherein the antibody is internalized into a cell after binding urokinase.

21. The antibody of claim 9, wherein the urokinase is bound to a urokinase cell surface receptor.

22. A pharmaceutical composition comprising the antibody of claim 1 or claim 13 and a pharmaceutically acceptable vehicle.

23. A diagnostic composition comprising the antibody of claim 1 or claim 18 and a pharmaceutically acceptable vehicle.

24. A method for inhibiting cell migration, cell invasion, cell proliferation or angiogenesis, comprising contacting cells with an effective amount of the antibody of claim 1 or claim 13.

25. A method for inhibiting cell migration, cell invasion, cell proliferation or angiogenesis, comprising contacting cells with an effective amount of the pharmaceutical composition of claim 22.

26. A method for treating or preventing cell migration, cell invasion, cell proliferation or angiogenesis in a patient comprising administering to the patient in need of such treatment a therapeutically effective amount of the antibody of claim 1 or claim 13.

27. A method for treating or preventing cell migration, cell invasion, cell proliferation or angiogenesis in a patient comprising administering to the patient in need of such treatment a therapeutically effective amount of the pharmaceutical composition of claim 22.

28. A method for inducing apoptosis comprising contacting cells with an effective amount of the antibody of claim 1 or claim 13.

29. A method for inducing apoptosis comprising contacting cells with an effective amount of the pharmaceutical composition of claim 22.

30. A method for inducing apoptosis in a patient comprising administering to a patient in need of such treatment a therapeutically effective amount of the antibody of claim 1 or claim 13.

31. A method for inducing apoptosis in a patient comprising administering to a patient in need of such treatment a therapeutically effective amount of the pharmaceutical composition of claim 22.

32. A method for treating or preventing a disease caused by cell migration, cell invasion, cell proliferation or angiogenesis in a patient comprising administering to the patient in need of such treatment a therapeutically effective amount of the antibody of claim 1 or claim 13.

33. A method for treating or preventing a disease caused by cell migration, cell invasion, cell proliferation or angiogenesis in a patient comprising administering to the patient in need of such treatment a therapeutically effective amount of the pharmaceutical composition of claim 22.

34. The method of claim 32, wherein the disease is primary growth of a solid tumor, leukemia or lymphoma; tumor invasion, metastasis or growth of tumor metastases; benign hyperplasia; atherosclerosis; myocardial angiogenesis; post-balloon angioplasty vascular restenosis; neointima formation following vascular trauma; vascular graft restenosis; coronary collateral formation; deep venous thrombosis; ischemic limb angiogenesis; telangiectasia; pyogenic granuloma; corneal disease; rubeosis; neovascular glaucoma; diabetic and other retinopathy; retrolental fibroplasia; diabetic neovascularization; macular degeneration; endometriosis; arthritis; fibrosis associated with a chronic inflammatory condition, traumatic spinal cord injury including ischemia, scarring or fibrosis; lung fibrosis, chemotherapy-induced fibrosis; wound healing with scarring and fibrosis; peptic ulcers; a bone fracture; keloids; or a disorder of vasculogenesis, hematopoiesis, ovulation, menstruation, pregnancy or placentation associated with pathogenic cell invasion or with angiogenesis.

35. The method of claim 33, wherein the disease is primary growth of a solid tumor, leukemia or lymphoma; tumor invasion, metastasis or growth of tumor metastases; benign hyperplasia; atherosclerosis; myocardial angiogenesis; post-balloon angioplasty vascular restenosis; neointima formation following vascular trauma; vascular graft restenosis; coronary collateral formation; deep venous thrombosis; ischemic limb angiogenesis; telangiectasia; pyogenic granuloma; corneal disease; rubeosis; neovascular glaucoma; diabetic and other retinopathy; retrolental fibroplasia; diabetic neovascularization; macular degeneration; endometriosis; arthritis; fibrosis associated with a chronic inflammatory condition, traumatic spinal cord injury including ischemia, scarring or fibrosis; lung fibrosis, chemotherapy-induced fibrosis; wound healing with scarring and fibrosis; peptic ulcers; a bone fracture; keloids; or a disorder of vasculogenesis, hematopoiesis, ovulation, menstruation, pregnancy or placentation associated with pathogenic cell invasion or with angiogenesis.

36. A method for detecting cell migration, cell invasion, cell proliferation or angiogenesis, comprising contacting cells with an effective amount of the antibody of claim 1 or claim 18.

37. A method for detecting cell migration, cell invasion, cell proliferation or angiogenesis, comprising contacting cells with an effective amount of the diagnostic composition of claim 23.

38. A method for detecting a disease caused by cell migration, cell invasion, cell proliferation or angiogenesis in a patient comprising administering to the patient in need of such treatment a diganostically effective amount of the antibody of claim 1 or claim 18.

39. A method for detecting a disease caused by cell migration, cell invasion, cell proliferation or angiogenesis in a patient comprising administering to the patient in need of such treatment a diganostically effective amount of the diagnostic composition of claim 23.

40. A method for detecting a disease caused by cell migration, cell invasion, cell proliferation or angiogenesis in a patient comprising administering to the patient in need of such treatment a diganostically effective amount of the antibody of claim 1 or claim 18.

41. A method for detecting whether the antibody of any one of claims 1, 9 or 13 is internalized into a cell comprising:
contacting the cell with the antibody;
washing, fixing and permeabilizing the cell;
adding a diagnostically labeled secondary antibody; and
detecting the diagnostic label.

42. A method for detecting whether the antibody of any one of claims 1, 9 or 13 is internalized into a cell comprising:
diagnostically labeling the antibody;
contacting the cell with the diagnostically labeled antibody; and
detecting the diagnostic label.


SEQUENCE LISTING (1 sequence)

SEQ ID NO: 1 - protein, 283 aa, Homo sapiens
LRCMQCKTNGDCRVEECALGQDLCRTTIVRLWEEGEELELVEKSCTHSEKTNRTLSYRTGLKITSLTEVVCGLDLCNQGNSGRAVTYSRSRYLECISCGSSDMSCERGRHQSLQCRSPEEQCLDVVTHWIQEGEEGRPKDDRHLRGCGYLPGCPGSNGFHNNDTFHFLKCCNTTKCNEGPILELENLPQNGRQCYSCKGNSTHGCSSEETFLIDCRGPMNQCLVATGTHEPKNQSYMVRGCATASMCQHAHLGDAFSMNHIDVSCCTKSGCNHPDLDVQYRSG